(19) United States
(12) Patent Application Publication
(10) Pub. No.: US 2021/0323993 A1
(43) Pub. Date: Oct. 21, 2021

(54) Compounds Useful in HIV Therapy

(71) Applicants: GlaxosmithKline Intellectual Property (No. 2) Limited, Brentford, Middlesex (GB); ViiV Healthcare Company, Wilmington, DE (US)

(72) Inventors: John MILLER, Research Triangle Park, NC (US); David TEMELKOFF, Research Triangle Park, NC (US); Emile Johann VELTHUISEN, Research Triangle Park, NC (US); Martha Alicia DE LA ROSA, Research Triangle Park, NC (US); Lisa SUWANDI, Research Triangle Park, NC (US)

(73) Assignees: GlaxosmithKline Intellectual Property (No. 2) Limited, Brentford, Middlesex (GB); ViiV Healthcare Company, Wilmington, DE (US)

(21) Appl. No.: 17/269,000

(22) PCT Filed: Aug. 28, 2019
(86) PCT No.: PCT/IB2019/057242

§ 371 Date: Feb. 17, 2021

Related U.S. Application Data
(60) Provisional application No. 62/724,647, filed on Aug. 30, 2018.

(51) Int. Cl. C07H 19/16; A61P 31/18

(52) U.S. Cl. CPC C07H 19/16; A61P 31/18

(57) ABSTRACT

The invention relates to compounds of Formulae (I), (Ia), (II) and (IIa), salts thereof, pharmaceutical compositions thereof, as well as methods of treating or preventing HIV in subjects.

### CROSS REFERENCE TO RELATED APPLICATIONS

The present application claims priority to U.S. Provisional Application Ser. No. 62/724,647 filed Aug. 30, 2018, the disclosure of which is incorporated herein by reference in its entirety.

### FIELD OF THE INVENTION

The present invention relates to compounds, pharmaceutical compositions, and methods of use thereof in connection with individuals infected with HIV.

### BACKGROUND OF THE INVENTION

Human immunodeficiency virus type 1 (HIV-1) infection leads to the contraction of acquired immune deficiency disease (AIDS). The number of cases of HIV continues to rise, and currently an estimated over thirty-five million individuals worldwide suffer from HIV infection e.g., http://www.sciencedirect.com/science/article/pii/S235230181630087X?via%3Dihub

Presently, long-term suppression of viral replication with antiretroviral drugs is the only option for treating HIV-1 infection. Indeed, the U.S. Food and Drug Administration has approved twenty-five drugs over six different inhibitor classes, which have been shown to greatly increase patient survival and quality of life. However, additional therapies are still believed to be required due to a number of issues including, but not limited to undesirable drug-drug interactions; drug-food interactions; non-adherence to therapy; drug resistance due to mutation of the enzyme target; and inflammation related to the immunologic damage caused by the HIV infection.

Currently, almost all HIV positive patients are treated with therapeutic regimens of antiretroviral drug combinations termed, highly active antiretroviral therapy (“HAART”). However, HAART therapies are often complex because a combination of different drugs must be administered often daily to the patient to avoid the rapid emergence of drug-resistant HIV-1 variants. Despite the positive impact of HAART on patient survival, drug resistance can still occur and the survival and quality of life are not normalized as compared to uninfected persons [Lohse Ann Intern Med 2007 146; 87-95]. Indeed, the incidence of several non-AIDS morbidities and mortalities, such as cardiovascular disease, frailty, and neurocognitive impairment, are increased in HAART-suppressed, HIV-infected subjects [Deeks Annu Rev Med 2011; 62:141-155]. This increased incidence of non-AIDS morbidity/mortality occurs in the context of, and is potentially caused by, elevated systemic inflammation related to the immunologic damage caused by HIV infection [Hunt J Infect Dis 2014][Byakagwa J Infect Dis 2014][Tenorio J Infect Dis 2014].

Modern antiretroviral therapy (ART) has the ability to effectively suppress HIV replication and improve health outcomes for HIV-infected persons, but is believed to not be capable of completely eliminating HIV viral reservoirs within the individual. HIV genomes can remain latent within mostly immune cells in the infected individual and may reactivate at any time, such that after interruption of ART, virus replication typically resumes within weeks. In a handful of individuals, the size of this viral reservoir has been significantly reduced and upon cessation of ART, the rebound of viral replication has been delayed [Henrich T J J Infect Dis 2013][Henrich T J Ann Intern Med 2014]. In one case, the viral reservoir was eliminated during treatment of leukemia and no viral rebound was observed during several years of follow-up [Nutter G N Engl J Med 2009]. These examples suggest the concept that reduction or elimination of the viral reservoir may be possible and can lead to viral remission or cure. As such, ways have been pursued to eliminate the viral reservoir, by direct molecular means, including excision of viral genomes with CRISPR/Cas9 systems, or to induce reactivation of the latent reservoir during ART so that the latent cells are eliminated. Induction of the latent reservoir typically results in either direct death of the latently infected cell or killing of the induced cell by the immune system after the virus is made visible. As this is performed during ART, viral genomes produced are believed to not result in the infection of new cells and the size of the reservoir may decay.

HAART therapies are often complex because a combination of different drugs must be administered often daily to the patient to avoid the rapid emergence of drug-resistant HIV-1 variants. Despite the positive impact of HAART on patient survival, drug resistance can still occur.

Current guidelines recommend that therapy includes three fully active drugs. See e.g. https://aidsinfo.nih.gov/quidelines

Typically, first-line therapies combine two to three drugs targeting the viral enzymes reverse transcriptase and integrase. It is believed that sustained successful treatment of HIV-1-infected patients with antiretroviral drugs employ the continued development of new and improved drugs that are effective against HIV strains that have formed resistance to approved drugs. For example, an individual on a regimen containing 3TC/FTC may select for the M184V mutation that reduces susceptibility to these drugs by >100 fold. See e g., https://hivdb.stanford.edu/dr-summary/resistance-notes/NRTI

Another way to potentially address preventing formation of mutations is to increase patient adherence to a drug regimen. One manner that may accomplish this is by reducing the dosing frequency. For parenteral administration, it is believed to be advantageous to have drug substances with high lipophilicity in order to reduce solubility and limit the release rate within interstitial fluid. However, most nucleoside reverse transcriptase inhibitors are hydrophilic thereby potentially limiting their use as long acting parenteral agents.

There remains a need for compounds which may the shortcomings set forth above.

### SUMMARY OF THE INVENTION

In one aspect, the invention provides a compound of the formula (I):

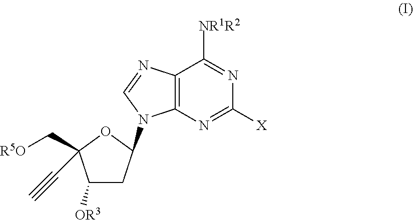

wherein:

R¹ and R² are each independently selected from the group consisting of H and —C(═O)—O—(C₁-C₆)alkyl

R³ is selected from the group consisting of H and —C(═O)—O—R⁴ wherein R⁴ is (C₁-C₆) alkyl;

R⁵ is selected from the group consisting of H,

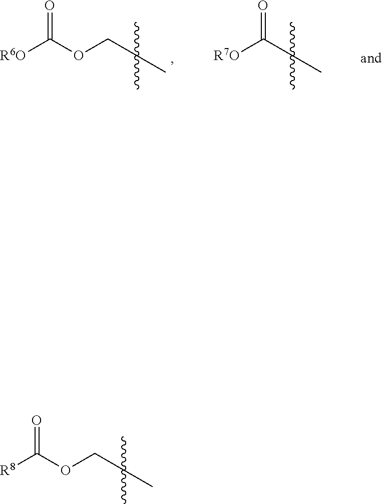

wherein R⁶, R⁷ and R⁸ are independently selected from (C₁-C₂₀)alkyl; and

X is selected from the group consisting of NH₂, F and Cl;

wherein when R³ and R⁵ are each H and X is F or Cl, both R¹ and R² cannot be H;

or a pharmaceutically acceptable salt thereof.

In another aspect, the invention provides a compound of the formula (II):

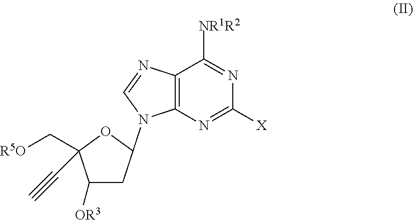

wherein:

R¹ and R² are each independently selected from the group consisting of H and —C(═O)—O—(C₁-C₆)alkyl;

R³ is selected from the group consisting of H and —C(═O)—O—R⁴ wherein R⁴ is (C₁-C₆) alkyl;

R⁵ is selected from the group consisting of H,

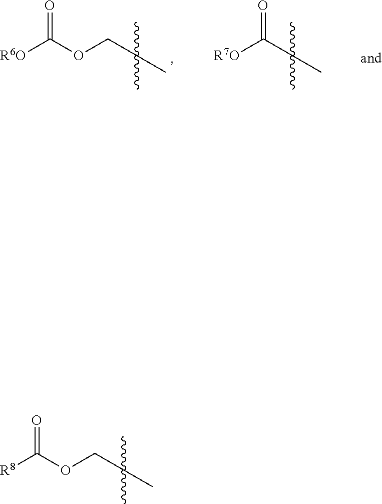

wherein R⁶, R⁷ and R⁸ are independently selected from (C₁-C₂₀)alkyl; and

X is selected from the group consisting of NH₂, F and Cl;

wherein when R³ and R⁵ are each H and X is F or Cl, both R¹ and R² cannot be H;

or a pharmaceutically acceptable salt thereof.

In another aspect, the invention provides pharmaceutical compositions comprising a compound of Formulas (I)-(II) a pharmaceutically acceptable salt thereof and an excipient

In another aspect, the invention provides a method of treating or preventing an HIV infection in a subject at risk for developing an HIV infection, comprising administering to the subject a compound of Formulas (I-(II), or a pharmaceutically acceptable salt thereof.

In another aspect, there is provided a compound of Formulas (I)-(II) or a pharmaceutically acceptable salt thereof for use in therapy.

In another aspect, there is provided a compound of Formulas (I)-(II) or a pharmaceutically acceptable salt thereof for use in treating or preventing an HIV infection.

In another aspect, there is provided the use of a compound of Formulas (I)-(II) or a pharmaceutically acceptable salt thereof in the manufacture of a medicament for treating or preventing an HIV infection.

These and other aspects are encompassed by the invention as set forth herein.

### BRIEF DESCRIPTION OF THE DRAWINGS

FIG. 1 illustrates mean whole blood concentration-time profiles of Compound 11 and EFdA after single muscular injection of Compound 11 at 20 mg/kg in male Wistar Han rats (N=3/time point); and

FIG. 2 illustrates mean whole blood concentration-time profiles of Compound 13 and EFdA after single muscular injection of Compound 13 at 20 mg/kg in male Wistar Han rats (N=3/time point).

### DETAILED DESCRIPTION OF REPRESENTATIVE EMBODIMENTS

Throughout this application, references are made to various embodiments relating to compounds, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

It is to be understood that the terminology used herein is for the purpose of describing particular embodiments only and is not intended to limit the scope of the present invention. In this specification and in the claims that follow, reference will be made to a number of terms that shall be defined to have the following meanings.

Where used herein the terms such as “a compound of formula (I), (Ia), (II) and (IIa)” and “compounds of formulae (I), (Ia), (II) and (IIa)” are intended to refer to each and all of the compounds (I), (Ia), (II) and (IIa) defined herein, i.e., the compounds of formulae (I), (Ia), (II) and (IIa).

“Alkyl” refers to a monovalent saturated aliphatic hydrocarbon group having from, e.g., 1 to 25 carbon, e.g., 1 to 10 carbon atoms atoms and, in some embodiments, from 1 to 6 carbon atoms. “(Cₓ-C_{y}) alkyl” refers to alkyl groups having from x to y carbon atoms. The term “alkyl” includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Alkylene” refers to divalent saturated aliphatic hydrocarbon groups that may having e.g., from 1 to 25 carbon atoms. The alkylene groups include branched and straight chain hydrocarbyl groups. For example, “(C₁-C₆)alkylene” is meant to include methylene, ethylene, propylene, 2-methypropylene, dimethylethylene, pentylene, and so forth. As such, the term “propylene” could be exemplified by the following structure:

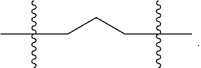 [SMILES: *CCC*]

Likewise, the term “dimethylbutylene” could be exemplified by any of the following three structures or more:

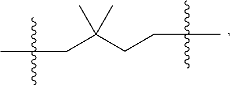 [SMILES: *CCC(C)(C)C*]

p or

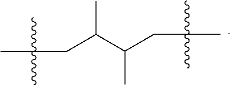 [SMILES: *CC(C)C(C)C*]

Furthermore, the term “(C₁-C₆)alkylene” is meant to include such branched chain hydrocarbyl groups as cyclopropylmethylene, which could be exemplified by the following structure:

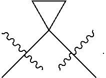 [SMILES: *C1(*)CC1]

“Alkenyl” refers to a linear or branched hydrocarbon group having, e.g., from 2 to 25, e.g., 2 to 20, e.g., 2 to 10 carbon atoms and in some embodiments from 2 to 6 carbon atoms or 2 to 4 carbon atoms and having at least 1 site of vinyl unsaturation (>C═C<). For example, (Cₓ-C_{y})alkenyl refers to alkenyl groups having from x to y carbon atoms and is meant to include for example, ethynyl, propenyl, isopropylene, 1,3-butadienyl, and the like Polyalkenyl substituents are also encompassed by this definition.

“Alkynyl” refers to a linear monovalent hydrocarbon radical or a branched monovalent hydrocarbon radical containing at least one triple bond. The term “alkynyl” is also meant to include those hydrocarbyl groups having one triple bond and one double bond. For example, (C₂-C₂₅), (C₂-C₂₀), or (C₂-C₆)alkynyl is meant to include ethynyl, propynyl, and the like. Polyalkynyl substituents are also encompassed by this definition.

“AUC” refers to the area under the plot of plasma concentration of drug (not logarithm of the concentration) against time after drug administration.

“EC₅₀” refers to the concentration of a drug that gives half-maximal response.

“IC₅₀” refers to the half-maximal inhibitory concentration of a drug. Sometimes, it is also converted to the pIC₅₀ scale (−log IC₅₀), in which higher values indicate exponentially greater potency.

“Haloalkyl” refers to substitution of an alkyl group with 1 to 3 halo groups (e.g., bifluoromethyl or trifluoromethyl).

“Compound”, “compounds”, “chemical entity”, and “chemical entities” as used herein refers to a compound encompassed by the generic formulae disclosed herein, any subgenus of those generic formulae, and any forms of the compounds within the generic and subgeneric formulae, including the racemates, stereoisomers, and tautomers of the compound or compounds.

The term “heteroatom” means nitrogen, oxygen, or sulfur and includes any oxidized form of nitrogen, such as N(O) {N⁺—O⁻} and sulfur such as S(O) and S(O)₂ and the quaternized form of any basic nitrogen.

“Polymorphism” refers to when two or more clearly different phenotypes exist in the same population of a species where the occurrence of more than one form or morph. In order to be classified as such, morphs must occupy the same habitat at the same time and belong to a panmictic population (one with random mating).

“Racemates” refers to a mixture of enantiomers. In an embodiment of the invention, the compounds of Formulas (I), (Ia), (II) and (IIa) or pharmaceutically acceptable salts thereof, are enantiomerically enriched with one enantiomer wherein all of the chiral carbons referred to are in one configuration. In general, reference to an enantiomerically enriched compound or salt, is meant to indicate that the specified enantiomer will comprise more than 50% by weight of the total weight of all enantiomers of the compound or salt.

“Solvate” or “solvates” of a compound refer to those compounds, as defined above, which are bound to a stoichiometric or non-stoichiometric amount of a solvent. Solvates of a compound includes solvates of all forms of the compound. In certain embodiments, solvents are volatile, non-toxic, and/or acceptable for administration to humans in trace amounts. Suitable solvates include water.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

The term ‘atropisomer’ refers to a stereoisomer resulting from an axis of asymmetry. This can result from restricted rotation about a single bond where the rotational barrier is high enough to allow differentiation of the isomeric species up to and including complete isolation of stable non-interconverting diastereomer or enantiomeric species. One skilled in the art will recognize that upon installing a nonsymmetrical R^{x} to core, the formation of atropisomers is possible. In addition, once a second chiral center is installed in a given molecule containing an atropisomer, the two chiral elements taken together can create diastereomeric and enantiomeric stereochemical species. Depending upon the substitution about the Cx axis, interconversion between the atropisomers may or may not be possible and may depend on temperature. In some instances, the atropisomers may interconvert rapidly at room temperature and not resolve under ambient conditions. Other situations may allow for resolution and isolation but interconversion can occur over a period of seconds to hours or even days or months such that optical purity is degraded measurably over time. Yet other species may be completely restricted from interconversion under ambient and/or elevated temperatures such that resolution and isolation is possible and yields stable species. When known, the resolved atropisomers were named using the helical nomenclature. For this designation, only the two ligands of highest priority in front and behind the axis are considered. When the turn priority from the front ligand 1 to the rear ligand 1 is clockwise, the configuration is P, if counterclockwise it is M.

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

“Patient” or “subject” refers to mammals and includes humans and non-human mammals.

Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

Where specific compounds or generic formulas are drawn that have aromatic rings, such as aryl or heteroaryl rings, then it will be understood by one of still in the art that the particular aromatic location of any double bonds are a blend of equivalent positions even if they are drawn in different locations from compound to compound or from formula to formula. For example, in the two pyridine rings (A and B) below, the double bonds are drawn in different locations, however, they are known to be the same structure and compound:

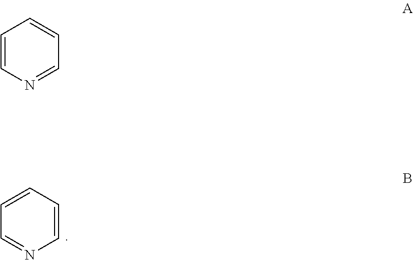 [SMILES: c1ccncc1 ; c1ccncc1]

The present invention includes compounds as well as their pharmaceutically acceptable salts. Accordingly, the word “or” in the context of “a compound or a pharmaceutically acceptable salt thereof” is understood to refer to either: 1) a compound alone or a compound and a pharmaceutically acceptable salt thereof (alternative), or 2) a compound and a pharmaceutically acceptable salt thereof (in combination).

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—. In a term such as “—C(R^{x})₂”, it should be understood that the two R^{x} groups can be the same, or they can be different if R^{x} is defined as having more than one possible identity. In addition, certain substituents are drawn as —R^{x}R^{y}, where the “—” indicates a bond adjacent to the parent molecule and R^{y} being the terminal portion of the functionality. Similarly, it is understood that the above definitions are not intended to include impermissible substitution patterns (e.g., methyl substituted with 5 fluoro groups). Such impermissible substitution patterns are well known to the skilled artisan.

In one aspect, the invention provides a compound of the formula (I):

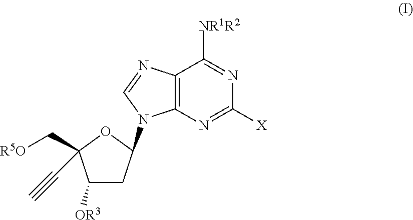

wherein:

R¹ and R² are each independently selected from the group consisting of H and —C(═O)—O—(C₁-C₆)alkyl

R³ is selected from the group consisting of H and —C(═O)—O—R⁴ wherein R⁴ is (C₁-C₆) alkyl;

R⁵ is selected from the group consisting of H,

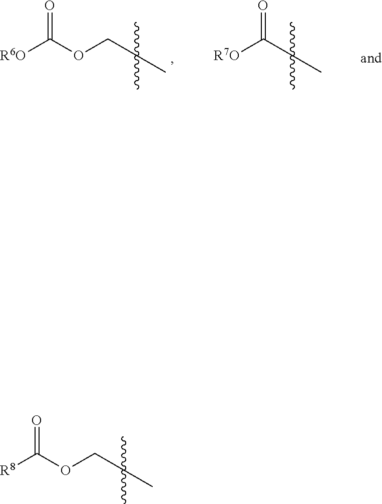

wherein R⁶, R⁷ and R⁸ are independently selected from (C₁-C₂₀)alkyl; and

X is selected from the group consisting of NH₂, F and C

wherein when R³ and R⁵ are each H and X is F or Cl, both R¹ and R² cannot be H;

or a pharmaceutically acceptable salt thereof.

In one aspect, the invention provides a compound of the formula (Ia):

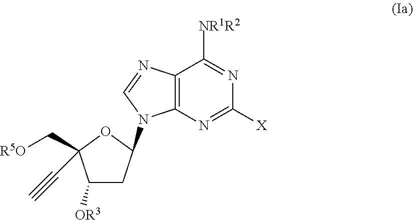

wherein:

R¹ and R² are each independently selected from the group consisting of H and —C(═O)—(C₁-C₂₅)alkyl;

R³ is selected from the group consisting of H, and —C(═O)—O—R⁴ wherein R⁴ is (C₁-C₂₅) alkyl;

R⁵ is selected from the group consisting of H,

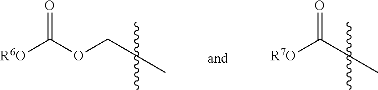

wherein R⁶ and R⁷ are independently selected from (C₁-C₂₅)alkyl; and

X is selected from the group consisting of NH₂, F and Cl;

wherein when R³ and R⁵ are each H and X is F or Cl, both R¹ and R² cannot be H;

or a pharmaceutically acceptable salt thereof.

In another aspect, the invention provides a compound of the formula (II):

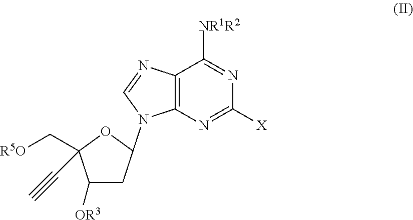

wherein:

R¹ and R² are each independently selected from the group consisting of H and —C(═O)—O—(C₁-C₆)alkyl

R³ is selected from the group consisting of H and —C(═O)—O—R⁴ wherein R⁴ is (C₁-C₆) alkyl;

R⁵ is selected from the group consisting of H,

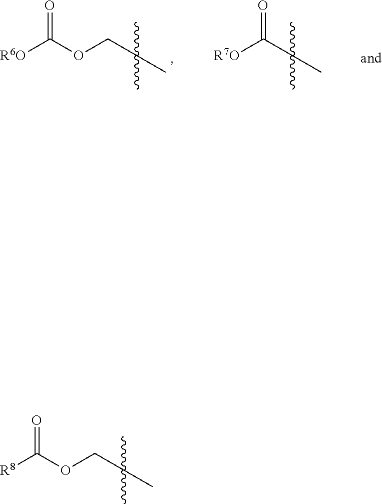

wherein R⁶, R⁷ and R⁸ are independently selected from (C₁-C₂₀)alkyl;

and X is selected from the group consisting of NH₂, F and Cl;

wherein when R³ and R⁵ are each H and X is F or Cl, both R¹ and R² cannot be H;

or a pharmaceutically acceptable salt thereof.

In one aspect, the invention provides a compound of the formula (IIa):

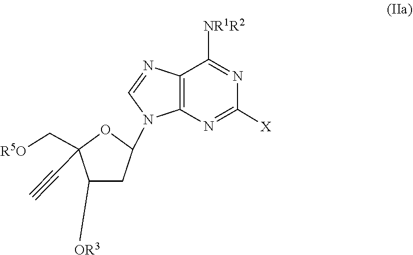

wherein:

R¹ and R² are each independently selected from the group consisting of H and —C(═O)—(C₁-C₂₅)alkyl;

R³ is selected from the group consisting of H, and —C(═O)—O—R⁴ wherein R⁴ is (C₁-C₂₅) alkyl;

R⁵ is selected from the group consisting of H,

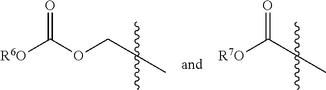

wherein R⁶ and R⁷ are independently selected from (C₁-C₂₅)alkyl; and

X is selected from the group consisting of NH₂, F and Cl;

wherein when R³ and R⁵ are each H and X is F or Cl, both R¹ and R² cannot be H;

or a pharmaceutically acceptable salt thereof.

Preferably in embodiments of formulae (I)-(II), R¹ and R² are each H.

Preferably in embodiments of formulae (I)-(II), R¹ is H and R² is —(C═O)—O—(C₁-C₆)alkyl.

Preferably in embodiments of formulae (I)-(II), X is F.

Preferably in embodiments of formulae (I)-(II), R³ is H.

Preferably in embodiments of formulae (I)-(II), R³ is:

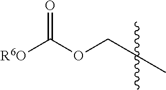

In various embodiments of formulae (I)-(II), R⁶ is (C₁-C15) alkyl.

In various embodiments of formulae (I)-(II), R⁶ is (C₁-C₁₀) alkyl.

In various embodiments of formulae (I)-(II), R⁶ is (C₁-C₆) alkyl.

Preferably in embodiments of formulae (I)-(II), R³ is:

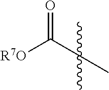

In various embodiments of formulae (I)-(II), R⁷ is (C₁-C₁₅)alkyl.

In various embodiments of formulae (I)-(II), R⁷ is (C₁-C₁₀) alkyl

In various embodiments of formulae (I)-(II), R⁷ is (C₁-C₆) alkyl

Preferably in embodiments of formulae (I)-(II), R₃ is:

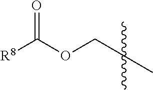

In various embodiments of formulae (I)-(II), R⁸ is (C₁-C₁₀) alkyl, more preferably (C₁-C₆) alkyl, most preferably C₃ alkyl.

In various embodiments of the formulae (Ia) and (IIa), X is F.

In various embodiments of the formulae (Ia) and (IIa), R¹ and R² are each H.

In various embodiments of the formulae (Ia) and (IIa), R¹ is H and R² is —C(═O)—(C₁-C₂₀)alkyl, more preferably, R² is —C(═O)—(C₁-C₁₅)alkyl.

In various embodiments of the formulae (Ia) and (IIa), R³ is H.

In various embodiments of the formulae (Ia) and (IIa), R³ is —C(═O)—O—R⁴ wherein R⁴ is (C₁-C₂₅) alkyl.

In various embodiments of the formulae (Ia) and (IIa), R⁵ is H.

In various embodiments of the formulae (Ia) and (IIa), R⁵ is:

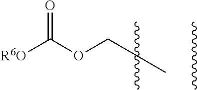

wherein R⁶ is selected from (C₁-C₂₅)alkyl. More preferably, R⁶ is selected from (C₁₅-C₂₀)alkyl.

In various embodiments of the formulae (Ia) and (IIa), R⁵ is:

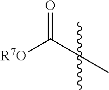

wherein R⁷ is selected from (C₁-C₂₅)alkyl. More preferably, R⁷ is selected from (C₁₅-C₂₀)alkyl

In another aspect of the present invention, the invention may encompass various individual compounds. As an example, such specific compounds may be selected from the group consisting of (Table 1):

TABLE 1

| Example | Structure | Chemical Name |
|---|---|---|
| 1 | 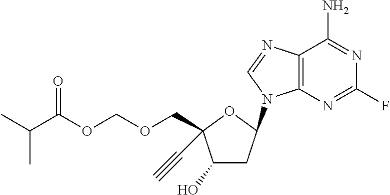 [SMILES: C#C[C@]1(COCOC(=O)C(C)C)O[C@@H](n2cnc3c(N)nc(F)nc32)C[C@@H]1O] | (((2R,35,5R)-5-(6-amino-2- fluoro-9H-purin-9-yl)-2-ethynyl- 3-hydroxytetrahydrofuran-2- yl)methoxy)methyl isobutyrate |
| 2 | 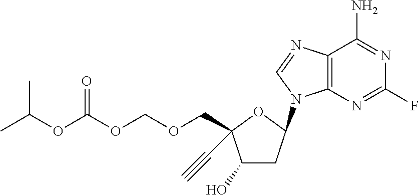 [SMILES: C#C[C@]1(COCOC(=O)OC(C)C)O[C@@H](n2cnc3c(N)nc(F)nc32)C[C@@H]1O] | (((2R,35,5R)-5-(6-amino-2- fluoro-9H-purin-9-yl)-2-ethynyl- 3-hydroxytetrahydrofuran-2- yl)methoxy)methyl isopropyl carbonate |
| 3 | 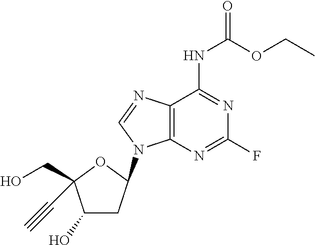 [SMILES: C#C[C@]1(CO)O[C@@H](n2cnc3c(NC(=O)OCC)nc(F)nc32)C[C@@H]1O] | ethyl (9-((2R,45,5R)-5-ethynyl- 4-hydroxy-5- (hydroxymethyl)tetrahydrofuran- 2-yl)-2-fluoro-9H-purin-6- yl)carbamate |
| 4 | 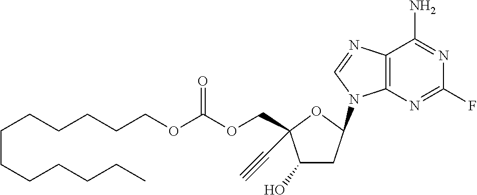 [SMILES: C#C[C@]1(COC(=O)OCCCCCCCCCCCC)O[C@@H](n2cnc3c(N)nc(F)nc32)C[C@@H]1O] | ((2R,35,5R)-5-(6-amino-2- fluoro-9H-purin-9-yl)-2-ethynyl- 3-hydroxytetrahydrofuran-2- yl)methyl dodecyl carbonate |
| 5 | 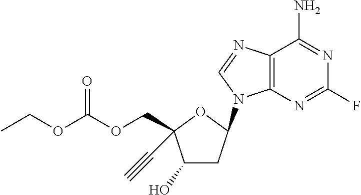 [SMILES: C#C[C@]1(COC(=O)OCC)O[C@@H](n2cnc3c(N)nc(F)nc32)C[C@@H]1O] | ((2R,35,5R)-5-(6-amino-2- fluoro-9H-purin-9-yl)-2-ethynyl- 3-hydroxytetrahydrofuran-2- yl)methyl ethyl carbonate |
| 6 | 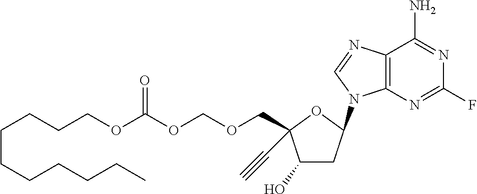 [SMILES: C#C[C@]1(COCOC(=O)OCCCCCCCCCC)O[C@@H](n2cnc3c(N)nc(F)nc32)C[C@@H]1O] | (((2R,3S,5R)-5-(6-amino-2- fluoro-9H-purin-9-yl)-2-ethynyl- 3-hydroxytetrahydrofuran-2- yl)methoxy)methyl decyl carbonate |
| 7 | 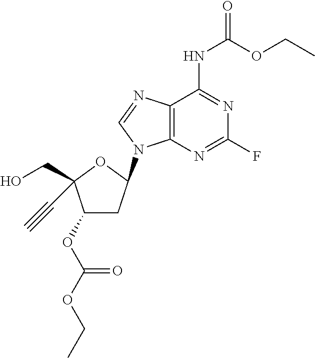 [SMILES: C#C[C@]1(CO)O[C@@H](n2cnc3c(NC(=O)OCC)nc(F)nc32)C[C@@H]1OC(=O)OCC] | ethyl (9-((2R,4S,5R)-4- ((ethoxycarbonyl)oxy)-5- ethynyl-5- (hydroxymethyl)tetrahydrofuran- 2-yl)-2-fluoro-9H-purin-6- yl)carbamate |
| 8 | 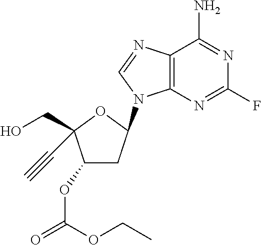 [SMILES: C#C[C@]1(CO)O[C@@H](n2cnc3c(N)nc(F)nc32)C[C@@H]1OC(=O)OCC] | (2R,3S,5R)-5-(6-amino-2-fluoro- 9H-purin-9-yl)-2-ethynyl-2- (hydroxymethyl)tetrahydrofuran- 3-yl ethyl carbonate |
| 9 | 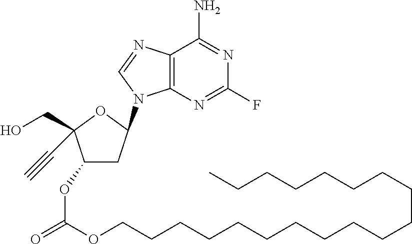 [SMILES: C#C[C@]1(CO)O[C@@H](n2cnc3c(N)nc(F)nc32)C[C@@H]1OC(=O)OCCCCCCCCCCCCCCCCCCC] | (2R,3S,5R)-5-(6-amino-2-fluoro- 9H-purin-9-yl)-2-ethynyl-2- (hydroxymethyl)tetrahydrofuran- 3-yl nonadecyl carbonate |
| 10 | 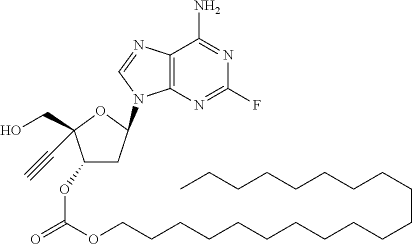 [SMILES: C#C[C@]1(CO)O[C@@H](n2cnc3c(N)nc(F)nc32)C[C@@H]1OC(=O)OCCCCCCCCCCCCCCCCCCCC] | (2R,3S,5R)-5-(6-amino-2-fluoro- 9H-purin-9-yl)-2-ethynyl-2- (hydroxymethyl)tetrahydrofuran- 3-yl icosyl carbonate |
| 11 | 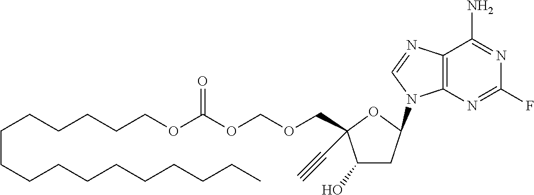 [SMILES: C#C[C@]1(COCOC(=O)OCCCCCCCCCCCCCCCC)O[C@@H](n2cnc3c(N)nc(F)nc32)C[C@@H]1O] | (((2R,3S,5R)-5-(6-amino-2- fluoro-9H-purin-9-yl)-2-ethynyl- 3-hydroxytetrahydrofuran-2- yl)methoxy)methyl hexadecyl carbonate |
| 12 | 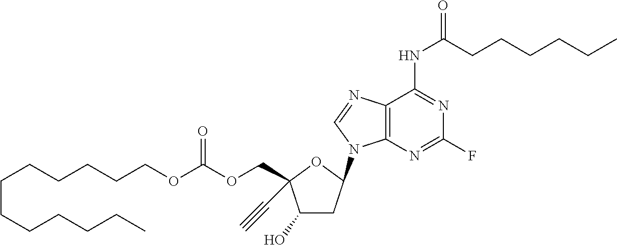 [SMILES: C#C[C@]1(COC(=O)OCCCCCCCCCCCC)O[C@@H](n2cnc3c(NC(=O)CCCCCC)nc(F)nc32)C[C@@H]1O] | dodecyl (((2R,3S,5R)-2-ethynyl- 5-(2-fluoro-6-heptanamido-9H- purin-9-yl)-3- hydroxytetrahydrofuran-2- yl)methyl) carbonate |
| 13 | 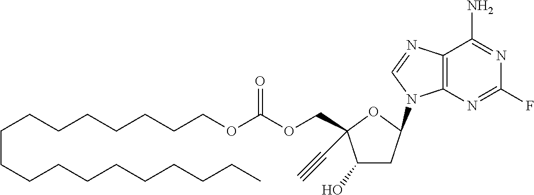 [SMILES: C#C[C@]1(COC(=O)OCCCCCCCCCCCCCCCCCC)O[C@@H](n2cnc3c(N)nc(F)nc32)C[C@@H]1O] | ((2R,3S,5R)-5-(6-amino-2- fluoro-9H-purin-9-yl)-2-ethynyl- 3-hydroxytetrahydrofuran-2- yl)methyl octadecyl carbonate |
| 14 | 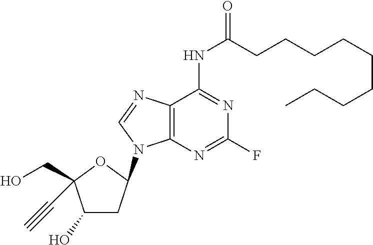 [SMILES: C#C[C@]1(CO)O[C@@H](n2cnc3c(NC(=O)CCCCCCCCC)nc(F)nc32)C[C@@H]1O] | N-(9-((2R,4S,5R)-5-ethynyl-4- hydroxy-5- (hydroxymethyl)tetrahydrofuran- 2-yl)-2-fluoro-9H-purin-6- yl)decanamide |
| 15 | 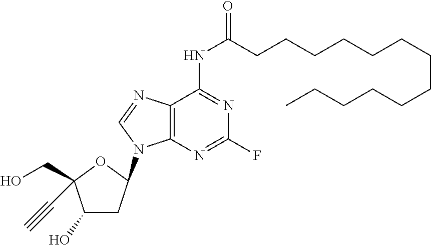 [SMILES: C#C[C@]1(CO)O[C@@H](n2cnc3c(NC(=O)CCCCCCCCCCCCC)nc(F)nc32)C[C@@H]1O] | N-(9-((2R,4S,5R)-5-ethynyl-4- hydroxy-5- (hydroxymethyl)tetrahydrofuran- 2-yl)-2-fluoro-9H-purin-6- yl)tetradecanamide |

and a pharmaceutically acceptable salt thereof.

In one embodiment, the present invention encompasses each individual compound listed in the above Table 1, or a pharmaceutically acceptable salt thereof.

In various embodiments, prodrugs of any of the compounds of formulae (I), (Ia), (II) and (IIa) set forth herein are also within the scope of the present invention.

In accordance with one embodiment of the present invention, there is provided a pharmaceutical composition comprising a compound of Formulae (I), (Ia), (II) and (IIa) or a pharmaceutically acceptable salt thereof, and a pharmaceutically acceptable excipient. In a further embodiment, the compound is present in amorphous form. In a further embodiment, the pharmaceutical composition is in a tablet form. In a further embodiment, the pharmaceutical composition is in parenteral form In a further embodiment, the compound is present as a spray dried dispersion.

In accordance with one embodiment of the present invention, there is provided a method of treating an HIV infection in a subject comprising administering to the subject a compound of Formulae (I), (Ia), (II) and (IIa) or a pharmaceutically acceptable salt thereof.

In accordance with one embodiment of the present invention, there is provided a method of treating an HIV infection in a subject comprising administering to the subject a pharmaceutical composition as described herein.

In accordance with one embodiment of the present invention, there is provided a method of preventing an HIV infection in a subject at risk for developing an HIV infection, comprising administering to the subject a compound of Formulae (I), (Ia), (II) and (IIa) or a pharmaceutically acceptable salt thereof.

In accordance with one embodiment of the present invention, there is provided the use of a compound of Formulae (I), (Ia), (II) and (IIa) in the manufacture of a medicament for treating an HIV infection.

In accordance with one embodiment of the present invention, there is provided the use of a compound of Formulae (I), (Ia), (II) and (IIa) in the manufacture of a medicament for preventing an HIV infection.

In accordance with one embodiment of the present invention, there is provided a compound according to Formulae (I), (Ia), (II) and (IIa) for use in treating an HIV infection.

In accordance with one embodiment of the present invention, there is provided a compound according to Formulae (I), (Ia), (II) and (IIa) for use in preventing an HIV infection.

In accordance with one embodiment of the present invention, there is provided a method of preventing an HIV infection in a subject at risk for developing an HIV infection, comprising administering to the subject a pharmaceutical composition as described herein.

Furthermore, the compounds of the invention can exist in particular geometric or stereoisomeric forms. The invention contemplates all such compounds, including cis- and trans-isomers, (−)- and (+)-enantiomers, (R)- and (S)-enantiomers, diastereomers, (D)-isomers, (L)-isomers, the racemic mixtures thereof, and other mixtures thereof, such as enantiomerically or diastereomerically enriched mixtures, as falling within the scope of the invention. Additional asymmetric carbon atoms can be present in a substituent such as an alkyl group. All such isomers, as well as mixtures thereof, are intended to be included in this invention.

Optically active (R)- and (S)-isomers and d and l isomers can be prepared using chiral synthons or chiral reagents, or resolved using conventional techniques. If, for instance, a particular enantiomer of a compound of the present invention is desired, it can be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as an amino group, or an acidic functional group, such as a carboxyl group, diastereomeric salts can be formed with an appropriate optically active acid or base, followed by resolution of the diastereomers thus formed by fractional crystallization or chromatographic means known in the art, and subsequent recovery of the pure enantiomers. In addition, separation of enantiomers and diastereomers is frequently accomplished using chromatography employing chiral, stationary phases, optionally in combination with chemical derivatization (e.g., formation of carbamates from amines).

In another embodiment of the invention, there is provided a compound of Formulae (I), (Ia), (II) and (IIa) wherein the compound or salt of the compound is used in the manufacture of a medicament for use in the treatment of an HIV infection in a human.

In another embodiment of the invention, there is provided a compound of Formulae (I), (Ia), (II) and (IIa) wherein the compound or salt of the compound is used in the manufacture of a medicament for use in the prevention of an HIV infection in a human.

In one embodiment, the pharmaceutical formulation containing a compound of Formulae (I), (Ia), (II) and (IIa) or a salt thereof is a formulation adapted for parenteral administration. In another embodiment, the formulation is a long-acting parenteral formulation. In a further embodiment, the formulation is a nano-particle formulation.

The compounds of the present invention and their salts, solvates, or other pharmaceutically acceptable derivatives thereof, may be employed alone or in combination with other therapeutic agents. Therefore, in other embodiments, the methods of treating and/or preventing an HIV infection in a subject may in addition to administration of a compound of Formulae (I), (Ia), (II) and (IIa) further comprise administration of one or more additional pharmaceutical agents active against HIV.

In such embodiments, the one or more additional agents active against HIV is selected from the group consisting of zidovudine, didanosine, lamivudine, zalcitabine, abacavir, stavudine, adefovir, adefovir dipivoxil, fozivudine, todoxil, emtricitabine, alovudine, amdoxovir, elvucitabine, nevirapine, delavirdine, efavirenz, loviride, immunocal, oltipraz, capravirine, lersivirine, GSK2248761, TMC-278, TMC-125, etravirine, saquinavir, ritonavir, indinavir, nelfinavir, amprenavir, fosamprenavir, brecanavir, darunavir, atazanavir, tipranavir, palinavir, lasinavir, enfuvirtide, T-20, T-1249, PRO-542, PRO-140, TNX-355, BMS-806, BMS-663068 and BMS-626529, 5-Helix, raltegravir, elvitegravir, dolutegravir, cabotegravir, vicriviroc (Sch-C), Sch-D, TAK779, maraviroc, TAK449, didanosine, tenofovir, lopinavir, and darunavir.

As such, the compounds of the present invention of Formulae (I), (Ia), (II) and (IIa) and any other pharmaceutically active agent(s) may be administered together or separately and, when administered separately, administration may occur simultaneously or sequentially, in any order. The amounts of the compounds of Formulae (I), (Ia), (II) and (IIa) of the present invention and the other pharmaceutically active agent(s) and the relative timings of administration will be selected in order to achieve the desired combined therapeutic effect. The administration in combination of a compound of the present invention of Formulae (I), (Ia), (II) and (IIa) and salts, solvates, or other pharmaceutically acceptable derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time. The amounts of the compound(s) of Formulae (I), (Ia), (II) and (IIa) or salts thereof and the other pharmaceutically active agent(s) and the relative timings of administration will be selected in order to achieve the desired combined therapeutic effect.

In addition, the compounds of the present invention of Formulae (I)-((I) may be used in combination with one or more other agents that may be useful in the prevention or treatmentof HIV. Examples of such agents include:
Nucleotide reverse transcriptase inhibitors such as zidovudine, didanosine, lamivudine, zalcitabine, abacavir, stavudine, adefovir, adefovir dipivoxil, fozivudine, todoxil, emtricitabine, alovudine, amdoxovir, elvucitabine, and similar agents;
Non-nucleotide reverse transcriptase inhibitors (including an agent having anti-oxidation activity such as immunocal, oltipraz, etc.) such as nevirapine, delavirdine, efavirenz, loviride, immunocal, oltipraz, capravirine, lersivirine, GSK2248761, TMC-278, TMC-125, etravirine, and similar agents;
Protease inhibitors such as saquinavir, ritonavir, indinavir, nelfinavir, amprenavir, fosamprenavir, brecanavir, darunavir, atazanavir, tipranavir, palinavir, lasinavir, and similar agents;
Entry, attachment and fusion inhibitors such as enfuvirtide (T-20), T-1249, PRO-542, PRO-140, TNX-355, BMS-806, BMS-663068, BMS-626529, 5-Helix and similar agents;
Inteqrase inhibitors such as raltegravir, elvitegravir, dolutegravir, bictegravir, cabotegravir and similar agents;
Maturation inhibitors such as PA-344 and PA-457, and similar agents; and
CXCR4 and/or CCR5 inhibitors such as vicriviroc (Sch-C), Sch-D, TAK779, maraviroc (UK 427,857), TAK449, as well as those disclosed in WO 02/74769, PCT/US03/39644, PCT/US03/39975, PCT/US03/39619, PCT/US03/39618, PCT/US03/39740, and PCT/US03/39732, and similar agents.

Other combinations may be used in conjunction with the compounds of the present invention, e.g., Biktarvy (Bictegravir/Emtricitabine/Tenofovir/Alafenamide) made commercially available by Gilead Sciences

Further examples where the compounds of the present invention may be used in combination with one or more agents useful in the prevention or treatment of HIV are found in Table 2.

TABLE 2

| FDA | Brand | Generic | |
|---|---|---|---|
| Approval | Name | Name | Manufacturer |
| Nucleoside Reverse | | | |
| Transcriptase | | | |
| Inhibitors (NRTIs) | | | |
| 1987 | Retrovir | zidovudine, | GlaxoSmithKline |
| | | azidothymidine, | |
| | | AZT, ZDV | |
| 1991 | Videx | didanosine, | Bristol-Myers |
| | | dideoxyinosine, | Squibb |
| | | ddl | |
| 1992 | Hivid | zalcitabine, | Roche |
| | | dideoxycytidine, | Pharmaceuticals |
| | | ddC | |
| 1994 | Zerit | stavudine, d4T | Bristol-Myers |
| | | | Squibb |
| 1995 | Epivir | lamivudine, 3TC | GlaxoSmithKline |
| 1997 | Combivir | lamivudine + | GlaxoSmithKline |
| | | zidovudine | |
| 1998 | Ziagen | abacavir sulfate, | GlaxoSmithKline |
| | | ABC | |
| 2000 | Trizivir | abacavir + | GlaxoSmithKline |
| | | lamivudine + | |
| | | zidovudine | |
| 2000 | Videx EC | enteric coated | Bristol-Myers |
| | | didanosine, ddl | Squibb |
| | | EC | |
| 2001 | Viread | tenofovir disoproxil | Gilead Sciences |
| | | fumarate, TDF | |
| 2003 | Emtriva | emtricitabine, FTC | Gilead Sciences |
| 2004 | Epzicom | abacavir + | GlaxoSmithKline |
| | | lamivudine | |
| 2004 | Truvada | emtricitabine + | Gilead Sciences |
| | | tenofovir disoproxil | |
| | | fumarate | |
| Non-Nucleosides | | | |
| Reverse Transcriptase | | | |
| Inhibitors (NNRTIs) | | | |
| 1996 | Viramune | nevirapine, NVP | Boehringer |
| | | | Ingelheim |
| 1997 | Rescriptor | delavirdine, DLV | Pfizer |
| 1998 | Sustiva | efavirenz, EFV | Bristol-Myers |
| | | | Squibb |
| 2008 | Intelence | Etravirine | Tibotec |
| | | | Therapeutics |
| Protease Inhibitors | | | |
| (PIs) | | | |
| 1995 | lnvirase | saquinavir | Roche |
| | | mesylate, SQV | Pharmaceuticals |
| 1996 | Norvir | ritonavir, RTV | Abbott |
| | | | Laboratories |
| 1996 | Crixivan | indinavir, IDV | Merck |
| 1997 | Viracept | nelfinavir | Pfizer |
| | | mesylate, NFV | |
| 1997 | Fortovase | saquinavir (no | Roche |
| | | longer marketed) | Pharmaceuticals |
| 1999 | Agenerase | amprenavir, APV | GlaxoSmithKline |
| 2000 | Kaletra | lopinavir+ ritonavir, | Abbott |
| | | LPV/RTV | Laboratories |
| 2003 | Reyataz | atazanavir sulfate, | Bristol-Myers |
| | | ATV | Squibb |
| 2003 | Lexiva | fosamprenavir | GlaxoSmithKline |
| | | calcium, FOS-APV | |
| 2005 | Aptivus | tripranavir, TPV | Boehringer |
| | | | Ingelheim |
| 2006 | Prezista | Darunavir | Tibotec |
| | | | Therapeutics |
| Fusion Inhibitors | | | |
| 2003 | Fuzeon | Enfuvirtide, T-20 | Roche |
| | | | Pharmaceuticals & |
| | | | Trimeris |
| Entry Inhibitors | | | |
| 2007 | Selzentry | Maraviroc | Pfizer |
| Integrase Inhibitors | | | |
| 2007 | Isentress | Raltegravir | Merck |
| 2013 | Tivicay | Dolutegravir | ViiV Healthcare |
| - - - | - - - | Cabotegravir | |

The scope of combinations of compounds of this invention with HIV agents is not limited to those mentioned above, but includes in principle any combination with any pharmaceutical composition useful for the treatment and/or prevention of HIV. As noted, in such combinations the compounds of the present invention and other HIV agents may be administered separately or in conjunction. In addition, one agent may be prior to, concurrent to, or subsequent to the administration of other agent(s).

The present invention may be used in combination with one or more agents useful as pharmacological enhancers as well as with or without additional compounds for the prevention or treatment of HIV. Examples of such pharmacological enhancers (or pharmakinetic boosters) include, but are not limited to, ritonavir, GS-9350, and SPI-452. Ritonavir is 10-hydroxy-2-methyl-5-(1-methyethyl)-1-1[2-(1-methylethyl)-4-thiazolyl]-3,6-dioxo-8,11-bis(phenylmethyl)-2,4,7,12-tetraazatridecan-13-oic acid, 5-thiazolylmethyl ester, [5S-(5S*,8R*,10R*,11R*)] and is available from Abbott Laboratories of Abbott park, Illinois, as Norvir. Ritonavir is an HIV protease inhibitor indicated with other antiretroviral agents for the treatment of HIV infection. Ritonavir also inhibits P450 mediated drug metabolism as well as the P-gycoprotein (Pgp) cell transport system, thereby resulting in increased concentrations of active compound within the organism.

GS-9350 is a compound being developed by Gilead Sciences of Foster City Calif. as a pharmacological enhancer.

SPI-452 is a compound being developed by Sequoia Pharmaceuticals of Gaithersburg, Md., as a pharmacological enhancer.

In one embodiment of the present invention, a compound of Formulae (I), (Ia), (II) and (IIa) is used in combination with ritonavir. In one embodiment, the combination is an oral fixed dose combination. In another embodiment, the compound of Formulae (I), (Ia), (II) and (IIa) is formulated as a long acting parenteral injection and ritonavir is formulated as an oral composition. In one embodiment, a kit containing the compound of Formulae (I), (Ia), (II) and (IIa) is formulated as a long acting parenteral injection and ritonavir formulated as an oral composition. In another embodiment, the compound of Formulae (I), (Ia), (II) and (IIa) is formulated as a long acting parenteral injection and ritonavir is formulated as an injectable composition. In one embodiment, a kit containing the compound of Formulae (I), (Ia), (II) and (IIa) is formulated as a long acting parenteral injection and ritonavir formulated as an injectable composition.

In another embodiment of the present invention, a compound of Formula (I), (Ia), (II) and (IIa) is used in combination with GS-9350. In one embodiment, the combination is an oral fixed dose combination. In another embodiment, the compound of Formulae (I), (Ia), (II) and (IIa) is formulated as a long acting parenteral injection and GS-9350 is formulated as an oral composition. In one embodiment, there is provided a kit containing the compound of Formulae (I), (Ia), (II) and (IIa) is formulated as a long acting parenteral injection and GS-9350 formulated as an oral composition. In another embodiment, the compound of Formulae (I), (Ia), (II) and (IIa) is formulated as a long acting parenteral injection and GS-9350 is formulated as an injectable composition. In one embodiment, is a kit containing the compound of Formulae (I), (Ia), (II) and (IIa) is formulated as a long acting parenteral injection and GS-9350 formulated as an injectable composition.

In one embodiment of the present invention, a compound of Formulae (I), (Ia), (II) and (IIa) is used in combination with SPI-452. In one embodiment, the combination is an oral fixed dose combination. In another embodiment, the compound of Formulae (I), (Ia), (II) and (IIa) is formulated as a long acting parenteral injection and SPI-452 is formulated as an oral composition. In one embodiment, there is provided a kit containing the compound of Formulae (I), (Ia), (II) and (IIa) formulated as a long acting parenteral injection and SPI-452 formulated as an oral composition. In another embodiment, the compound of Formulae (I), (Ia), (II) and (IIa) is formulated as a long acting parenteral injection and SPI-452 is formulated as an injectable composition. In one embodiment, there is provided a kit containing the compound of Formulae (I), (Ia), (II) and (IIa) formulated as a long acting parenteral injection and SPI-452 formulated as an injectable composition.

In one embodiment of the present invention, a compound of Formulae (I), (Ia), (II) and (IIa) is used in combination with compounds which are found in previously filed PCT/CN2011/0013021, which is herein incorporated by reference.

The above other therapeutic agents, when employed in combination with the chemical entities described herein, may be used, for example, in those amounts indicated in the Physicians' Desk Reference (PDR) or as otherwise determined by one of ordinary skill in the art.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formulae (I), (Ia), (II) and (IIa).

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formulae (I), (Ia), (II) and (IIa), wherein said virus is an HIV virus. In some embodiments, the HIV virus is the HIV-1 virus.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formulae (I), (Ia), (II) and (IIa) further comprising administration of a therapeutically effective amount of one or more agents active against an HIV virus.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formula (I), further comprising administration of a therapeutically effective amount of one or more agents active against the HIV virus, wherein said agent active against HIV virus is selected from Nucleotide reverse transcriptase inhibitors; Non-nucleotide reverse transcriptase inhibitors; Protease inhibitors; Entry, attachment and fusion inhibitors; Integrase inhibitors; Maturation inhibitors; CXCR4 inhibitors; and CCR5 inhibitors.

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formulae (I), (Ia), -(II) and (IIa).

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formulae (I), (Ia), (II) and (IIa), wherein said virus is an HIV virus. In some embodiments, the HIV virus is the HIV-1 virus.

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formulae (I), (Ia), (II) and (IIa), further comprising administration of a therapeutically effective amount of one or more agents active against an HIV virus.

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formulae (I), (Ia), (II) and (IIa) further comprising administration of a therapeutically effective amount of one or more agents active against the HIV virus, wherein said agent active against HIV virus is selected from Nucleotide reverse transcriptase inhibitors; Non-nucleotide reverse transcriptase inhibitors; Protease inhibitors; Entry, attachment and fusion inhibitors; Integrase inhibitors; Maturation inhibitors; CXCR4 inhibitors; and CCR5 inhibitors.

In further embodiments, the compound of the present invention of Formulae (I), (Ia), (II) and (IIa) or a pharmaceutically acceptable salt thereof, is selected from the group of compounds set forth in Table 1 above.

The compounds of Table 1 were synthesized according to the Synthetic Methods, General Schemes, and the Examples described below.

In another embodiment, there is provided a pharmaceutical composition comprising a pharmaceutically acceptable diluent and a therapeutically effective amount of a compound of Formulae (I), (Ia), (II) and (IIa) or a pharmaceutically acceptable salt thereof.

In certain embodiments, the compound(s) of the present invention, or a pharmaceutically acceptable salt thereof, is chosen from the compounds set forth in Table 1. The compounds of the present invention can be supplied in the form of a pharmaceutically acceptable salt. The terms “pharmaceutically acceptable salt” refer to salts prepared from pharmaceutically acceptable inorganic and organic acids and bases. Accordingly, the word “or” in the context of “a compound or a pharmaceutically acceptable salt thereof” is understood to refer to either a compound or a pharmaceutically acceptable salt thereof (alternative), or a compound and a pharmaceutically acceptable salt thereof (in combination).

As used herein, the term “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication. The skilled artisan will appreciate that pharmaceutically acceptable salts of compounds according to Formulae (I), (Ia), (II) and (IIa) may be prepared. These pharmaceutically acceptable salts may be prepared in situ during the final isolation and purification of the compound, or by separately reacting the purified compound in its free acid or free base form with a suitable base or acid, respectively.

Illustrative pharmaceutically acceptable acid salts of the compounds of the present invention can be prepared from the following acids, including, without limitation formic, acetic, propionic, benzoic, succinic, glycolic, gluconic, lactic, maleic, malic, tartaric, citric, nitic, ascorbic, glucuronic, maleic, fumaric, pyruvic, aspartic, glutamic, benzoic, hydrochloric, hydrobromic, hydroiodic, isocitric, trifluoroacetic, pamoic, propionic, anthranilic, mesylic, oxalacetic, oleic, stearic, salicylic, p-hydroxybenzoic, nicotinic, phenylacetic, mandelic, embonic (pamoic), methanesulfonic, phosphoric, phosphonic, ethanesulfonic, benzenesulfonic, pantothenic, toluenesulfonic, 2-hydroxyethanesulfonic, sulfanilic, sulfuric, salicylic, cyclohexylaminosulfonic, algenic, β-hydroxybutyric, galactaric and galacturonic acids. Preferred pharmaceutically acceptable salts include the salts of hydrochloric acid and trifluoroacetic acid.

Illustrative pharmaceutically acceptable inorganic base salts of the compounds of the present invention include metallic ions. More preferred metallic ions include, but are not limited to, appropriate alkali metal salts, alkaline earth metal salts and other physiological acceptable metal ions. Salts derived from inorganic bases include aluminum, ammonium, calcium, copper, ferric, ferrous, lithium, magnesium, manganic salts, manganous, potassium, sodium, zinc, and the like and in their usual valences. Exemplary base salts include aluminum, calcium, lithium, magnesium, potassium, sodium and zinc. Other exemplary base salts include the ammonium, calcium, magnesium, potassium, and sodium salts. Still other exemplary base salts include, for example, hydroxides, carbonates, hydrides, and alkoxides including NaOH, KOH, Na₂CO₃, K₂CO₃, NaH, and potassium-t-butoxide.

Salts derived from pharmaceutically acceptable organic non-toxic bases include salts of primary, secondary, and tertiary amines, including in part, trimethylamine, diethylamine, N,N′-dibenzylethylenediamine, chloroprocaine, choline, diethanolamine, ethylenediamine, meglumine (N-methylglucamine) and procaine; substituted amines including naturally occurring substituted amines; cyclic amines; quaternary ammonium cations; and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

All of the above salts can be prepared by those skilled in the art by conventional means from the corresponding compound of the present invention. For example, the pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. The salt may precipitate from solution and be collected by filtration or may be recovered by evaporation of the solvent. The degree of ionisation in the salt may vary from completely ionised to almost non-ionised. Lists of suitable salts are found in Remington's Pharmaceutical Sciences, 17th ed., Mack Publishing Company, Easton, Pa., 1985, p.1418, the disclosure of which is hereby incorporated by reference only with regards to the lists of suitable salts.

The compounds of Formulae (I), (Ia), (II) and (IIa) of the invention may exist in both unsolvated and solvated forms. The term ‘solvate’ comprises the compound of the invention and one or more pharmaceutically acceptable solvent molecules, for example, ethanol. The term ‘hydrate’ is employed when said solvent is water. Pharmaceutically acceptable solvates include hydrates and other solvates wherein the solvent of crystallization may be isotopically substituted, e.g. D₂O, d₆-acetone, d₆-DMSO.

Compounds of Formulae (I), (Ia), (II) and (IIa) containing one or more asymmetric carbon atoms can exist as two or more stereoisomers. Where a compound of Formulae (I), (Ia), (II) and (IIa) contains an alkenyl or alkenylene group or a cycloalkyl group, geometric cis/trans (or Z/E) isomers are possible. Where the compound contains, for example, a keto or oxime group or an aromatic moiety, tautomeric isomerism (‘tautomerism’) can occur. It follows that a single compound may exhibit more than one type of isomerism.

Included within the scope of the claimed compounds present invention are all stereoisomers, geometric isomers and tautomeric forms of the compounds of Formulae (I), (Ia), (II) and (IIa), including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of Formulae (I), (Ia), (II) and (IIa) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Mixtures of stereoisomers may be separated by conventional techniques known to those skilled in the art. [see, for example, “Stereochemistry of Organic Compounds” by E L Eliel (Wiley, New York 1994).]

The present invention includes all pharmaceutically acceptable isotopically-labelled compounds of Formulae (I), (Ia), (II) and (IIa) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of Formulae (I), (Ia), (II) and (IIa), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Isotopically-labelled compounds of Formulae (I), (Ia), (II) and (IIa) can generally be prepared by conventional techniques known to those skilled in the art or by processes analogous to those described herein using an appropriate isotopically-labelled reagents in place of the non-labelled reagent previously employed.

The compounds of the present invention may be administered as prodrugs. In one embodiment, the compounds of the invention are prodrugs of 4′-ethylnyl-2-fluoro-2′-deoxyadenosine (EFdA) disclosed e.g., in U.S. Pat. No. 7,339,053, which is a nucleoside reverse transcriptase inhibitor of the formula:

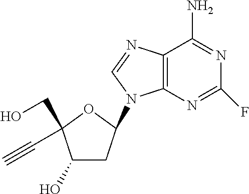 [SMILES: C#C[C@]1(CO)O[C@@H](n2cnc3c(N)nc(F)nc32)C[C@@H]1O]

The prodrugs are useful in that they are capable of modulating physicochemical properties, facilitating multiple dosing paradigms and improving pharmacokinetic and/or pharmacodynamic profiles of the active parent (EfdA). More specifically, EFdA has a relatively high aqueous solubility, rendering it unsuitable for slow release, long acting, parenteral dosing. Advantageously, prodrugs of EFdA of the invention are capable of having substantially reduced aqueous solubilities, that in some cases, may facilitate a slow release, parenteral dosing modality. Additionally, prodrugs of EFdA, of the invention may also reduce or eliminate undesirable injection site reactions associated with high localized concentrations of EFdA that occur upon parenteral dosing of EFdA itself. Moreover, prodrugs of EFdA of the invention may also, in some cases, confer an enhancement in antiviral persistence as compared to EFdA.

Administration of the chemical entities and combinations of entities described herein can be via any of the accepted modes of administration for agents that serve similar utilities including, but not limited to, orally, sublingually, subcutaneously, intravenously, intranasally, topically, transdermally, intraperitoneally, intramuscularly, intrapulmonarilly, vaginally, rectally, or intraocularly. In some embodiments, oral or parenteral administration is used. Examples of dosing include, without limitation, once every seven days for oral, once every eight weeks for intramuscular, or once every six months for subcutaneous.

Pharmaceutical compositions or formulations include solid, semi-solid, liquid and aerosol dosage forms, such as, e.g., tablets, capsules, powders, liquids, suspensions, suppositories, aerosols or the like. The chemical entities can also be administered in sustained or controlled release dosage forms, including depot injections, osmotic pumps, pills, transdermal (including electrotransport) patches, and the like, for prolonged and/or timed, pulsed administration at a predetermined rate. In certain embodiments, the compositions are provided in unit dosage forms suitable for single administration of a precise dose.

The chemical entities described herein can be administered either alone or more typically in combination with a conventional pharmaceutical carrier, excipient or the like (e.g., mannitol, lactose, starch, magnesium stearate, sodium saccharine, talcum, cellulose, sodium crosscarmellose, glucose, gelatin, sucrose, magnesium carbonate, and the like). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate, and the like). Generally, depending on the intended mode of administration, the pharmaceutical composition will contain about 0.005% to 95%; in certain embodiments, about 0.5% to 50% by weight of a chemical entity. Actual methods of preparing such dosage forms are known, or will be apparent, to those skilled in this art; for example, see Remington's Pharmaceutical Sciences, Mack Publishing Company, Easton, Pa.

In certain embodiments, the compositions will take the form of a pill or tablet and thus the composition will contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils or triglycerides) is encapsulated in a gelatin capsule.

Liquid pharmaceutically administrable compositions can, for example, be prepared by dissolving, dispersing, etc. at least one chemical entity and optional pharmaceutical adjuvants in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of chemical entities contained in such parenteral compositions is highly dependent on the specific nature thereof, as well as the activity of the chemical entities and the needs of the subject. However, percentages of active ingredient of 0.01% to 10% in solution are employable; and will be higher if the composition is a solid which will be subsequently diluted to the above percentages. In certain embodiments, the composition may comprise from about 0.2 to 2% of the active agent in solution.

Pharmaceutical compositions of the chemical entities described herein may also be administered to the respiratory tract as an aerosol or solution for a nebulizer, or as a microfine powder for insufflation, alone or in combination with an inert carrier such as lactose. In such a case, the particles of the pharmaceutical composition have diameters of less than 50 microns, in certain embodiments, less than 10 microns.

In general, the chemical entities provided will be administered in a therapeutically effective amount by any of the accepted modes of administration for agents that serve similar utilities. The actual amount of the chemical entity, i.e., the active ingredient, will depend upon numerous factors such as the severity of the disease to be treated, the age and relative health of the subject, the potency of the chemical entity used the route and form of administration, and other factors. The drug can be administered more than once a day, such as once or twice a day.

In general, the chemical entities will be administered as pharmaceutical compositions by any one of the following routes: oral, systemic (e.g., transdermal, intranasal or by suppository), or parenteral (e.g., intramuscular, intravenous or subcutaneous) administration. In certain embodiments, oral administration with a convenient daily dosage regimen that can be adjusted according to the degree of affliction may be used. Compositions can take the form of tablets, pills, capsules, semisolids, powders, sustained release formulations, solutions, suspensions, elixirs, aerosols, or any other appropriate compositions. Another manner for administering the provided chemical entities is inhalation.

The choice of formulation depends on various factors such as the mode of drug administration and bioavailability of the drug substance. For delivery via inhalation the chemical entity can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDIs typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical compositions have been developed for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a cross-linked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of, in general, at least one chemical entity described herein in combination with at least one pharmaceutically acceptable excipient. Acceptable excipients are non-toxic, aid administration, and do not adversely affect the therapeutic benefit of the at least one chemical entity described herein. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, for injectable solutions, include water, saline, aqueous dextrose, and glycols.

Compressed gases may be used to disperse a chemical entity described herein in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the chemical entity in a composition can vary within the full range employed by those skilled in the art. Typically, the composition will contain, on a weight percent (wt %) basis, from about 0.01-99.99 wt % of at least one chemical entity described herein based on the total composition, with the balance being one or more suitable pharmaceutical excipients. In certain embodiments, the at least one chemical entity described herein is present at a level of about 1-80 wt %.

In various embodiments, pharmaceutical compositions of the present invention encompass compounds of Formulae (I), (Ia), (II) and (IIa), salts thereof, and combinations of the above.

### Synthetic Methods

The methods of synthesis may employ readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

Additionally, the methods of this invention may employ protecting groups which prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the provided chemical entities may contain one or more chiral centers and such compounds can be prepared or isolated as pure stereoisomers, i.e., as individual enantiomers or diastereomers, or as stereoisomer-enriched mixtures. All such stereoisomers (and enriched mixtures) are included within the scope of this specification, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Ernka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4th Edition), and Larock's Comprehensive Organic Transformations (VCH Publishers Inc., 1989).

Unless specified to the contrary, the reactions described herein may or take place at atmospheric pressure, generally within a temperature range from −78° C. to 200° C. Further, except as employed in the Example or as otherwise specified, reaction times and conditions are intended to be approximate, e.g., taking place at about atmospheric pressure within a temperature range of about −78° C. to about 110° C. over a period of about 1 to about 24 hours; reactions left to run overnight average a period of about 16 hours.

The terms “solvent,” “organic solvent,” and “inert solvent” each mean a solvent inert under the conditions of the reaction being described in conjunction therewith, including, for example, benzene, toluene, acetonitrile, tetrahydrofuranyl (“THF”), dimethylformamide (“DMF”), chloroform, methylene chloride (or dichloromethane), diethyl ether, methanol, N-methylpyrrolidone (“NMP”), pyridine and the like.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When desired, the (R)- and (S)-isomers may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. Alternatively, a specific enantiomer may be synthesized by asymmetric synthesis using optically active reagents, substrates, catalysts or solvents, or by converting one enantiomer to the other by asymmetric transformation.

### EXAMPLES AND GENERAL SYNTHESIS

The following examples and prophetic synthesis method serve to more fully describe the manner of making and using the above-described invention. It is understood that this in no way serve to limit the true scope of the invention, but rather is presented for illustrative purposes. Unless otherwise specified, the following abbreviations have the following meanings. If an abbreviation is not defined, it has its generally accepted meaning.
aq.=aqueous
μL=microliters
μM=micromolar
NMR=nuclear magnetic resonance
Boc=tert-butoxycarbonyl
br=broad
Cbz=benzyloxycarbonyl
d=doublet
° C.=degrees Celsius
DCM=dichloromethane
dd=doublet of doublets
DIEA =N,N-diisopropylethylamine
DMAP=N,N-dimethylaminopyridine
DMEM32 Dulbeco's Modified Eagle's Medium
DMF=N,N-dimethylformamide
DMSO=dimethylsufoxide
EDC=N-(3-Dimethylaminopropyl)-N′-ethylcarbodiimide hydrochloride
EtOAc=ethyl acetate
g=gram
h or hr=hour(s)
HPLC=high performance liquid chromatography
Hz=Hertz
IU=International Units
IC₅₀=50% inhibitory concentration
J=coupling constant in Hz
LCMS=liquid chromatography mass spectrometry
m=multiplet
M=molar concentration
M+H⁺=parent mass spectrum peak plus H⁺
mg=miligram
min=minute(s)
mL=milliliter
mM=millimolar
mmol=millimole
MMTr=monomethoxytrityl
MS=mass spectrum
MTBE=methyl tert-butyl ether
nM=nanomolar
PE=petroleum ether
ppm=parts per million
q.s.=sufficient amount
s=singlet
RT=room temperature
sat.=saturated
t=triplet
TBDMS=tert-butyldimethylsilyl
TBDPS=tert-butyldiphenylsilyl
TEA=triethylamine
THF=tetrahydrofuran
TMS=trimethylsilyl

Additionally, various compounds of the invention may be made, in one embodiment, by way of the general synthesis routes set forth in Schemes 1 and 2 below:

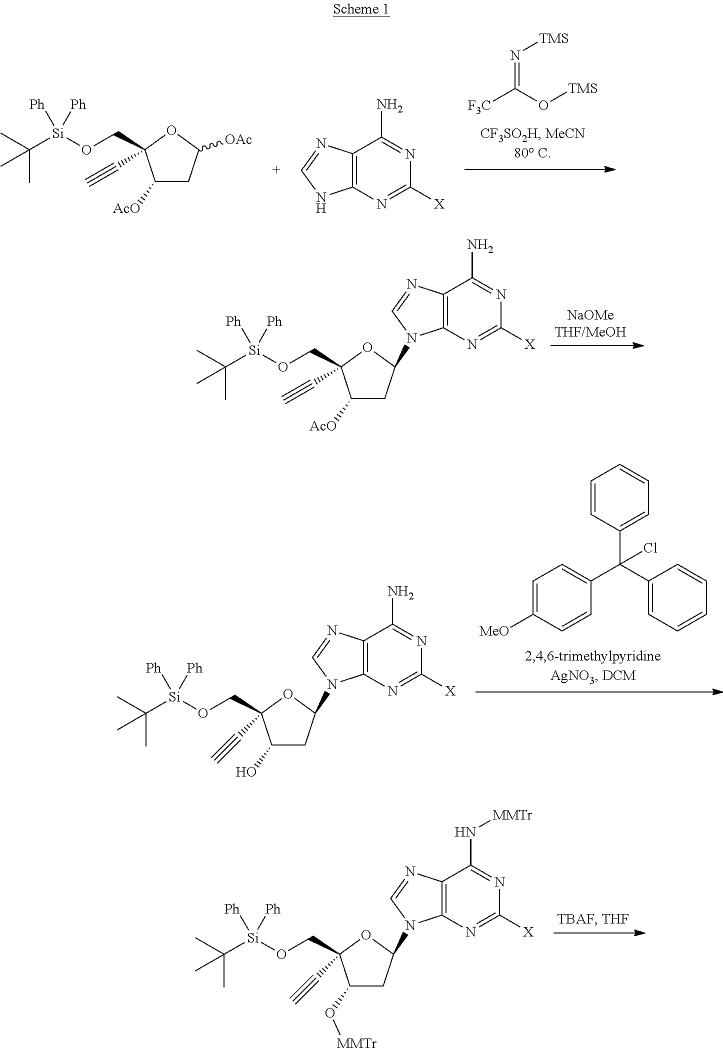

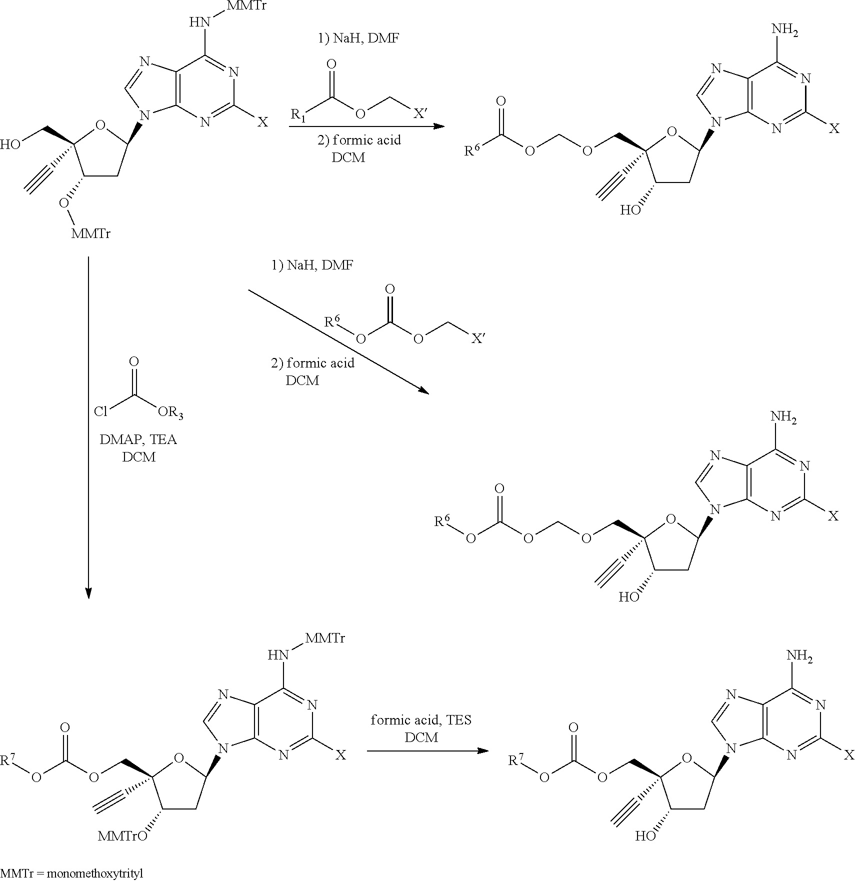

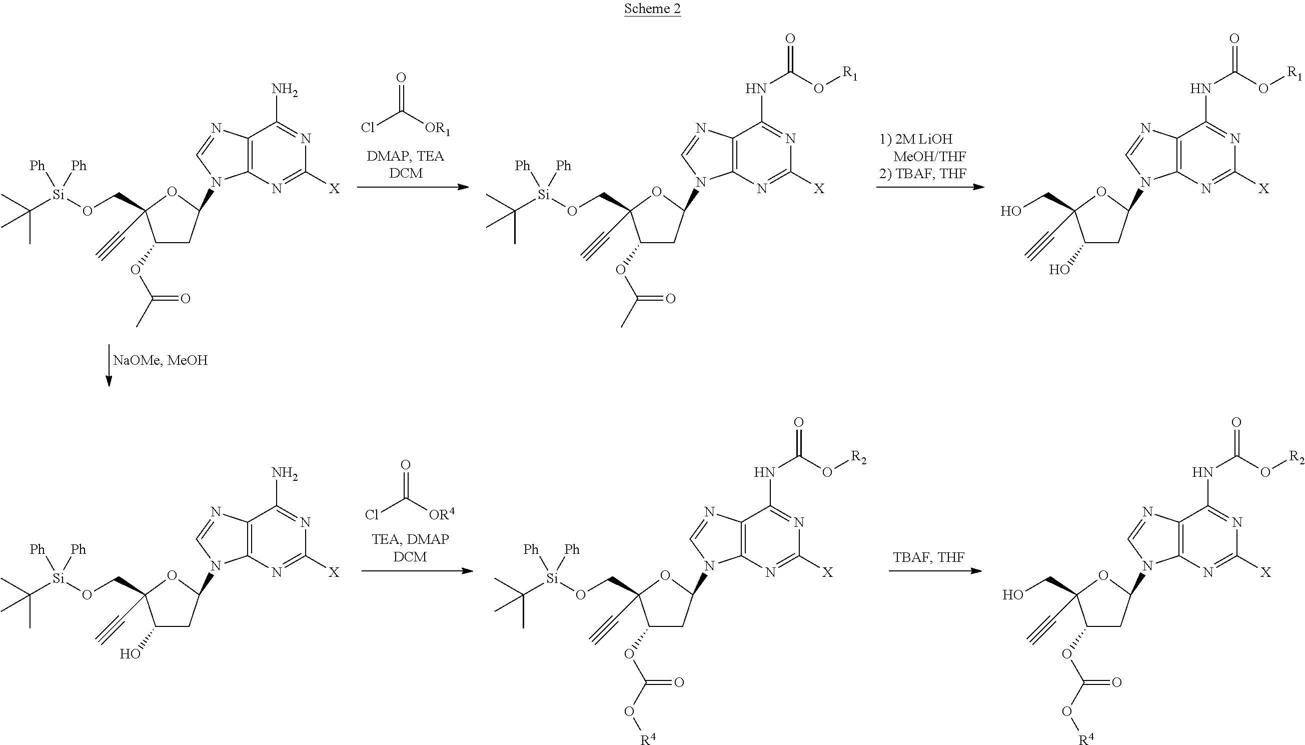

wherein R⁴ ,R⁶ and R⁷ are defined hereinabove.
wherein:
Ac=acetyl
AcO=acetate
AgNO₃=silver nitrate
DCM=dichloromethane
DMAP=4-dimethylaminopyridine
DMF=N,N-dimethylformamide
MeCN=acetonitrile
MeOH=methanol
NaH=sodium hydride
NaOMe=sodium methoxide
Ph=phenyl
TEA=triethylamine
TES=triethylsilane
TBAF=tetra-n-butyl ammonium fluoride
THF=tetrahydrofuran
X′=halogen
and wherein X and R-groups are defined hereinabove

### Equipment Description

¹H NMR spectra were recorded on Varian or Bruker spectrometers. Chemical shifts are expressed in parts per million (ppm, 6 units). Coupling constants are in units of hertz (Hz). Splitting patterns describe apparent multiplicities and are designated as s (singlet), d (doublet), t (triplet), q (quartet), quint (quintet), m (multiplet), br (broad).

Representative equipment and conditions for acquiring analytical low resolution LCMS are described below.

### Instrumentation:

Waters Acquity UPLC-MS system with SQ detectors

### MS Conditions:

Scan Mode: Alternating positive/negative electrospray
Scan Range: 125-1200 amu
Scan Time: 150 msec
Interscan Delay: 50 msec

### LC Conditions:

The UPLC analysis was conducted on a Phenomenex Kinetex 1.7 um 2.1×50mm XB-C18 column at 40° C.
0.2 uL of sample was injected using PLNO (partial loop with needle overfill) injection mode.
The gradient employed was:
Mobile Phase A: Water+0.2% v/v Formic Acid
Mobile Phase B: Acetonitrile+0.15% v/v Formic Acid

| Time | % A | % B | Flow Rate |
|---|---|---|---|
| 0.00 min | 95 | 5 | 1 ml/min |
| 1.1 min | 1 | 99 | 1 ml/min |
| 1.5 min | 1 | 99 | 1 ml/min |

UV detection provided by summed absorbance signal from 210 to 350 nm scanning at 40 Hz

### Example 1

### (((2R,3S,5R)-5-(6-amino-2-fluoro-9H-purin-9-yl)-2-ethynyl-3-hydroxytetrahydrofuran-2-yl)methoxy)methyl isobutyrate

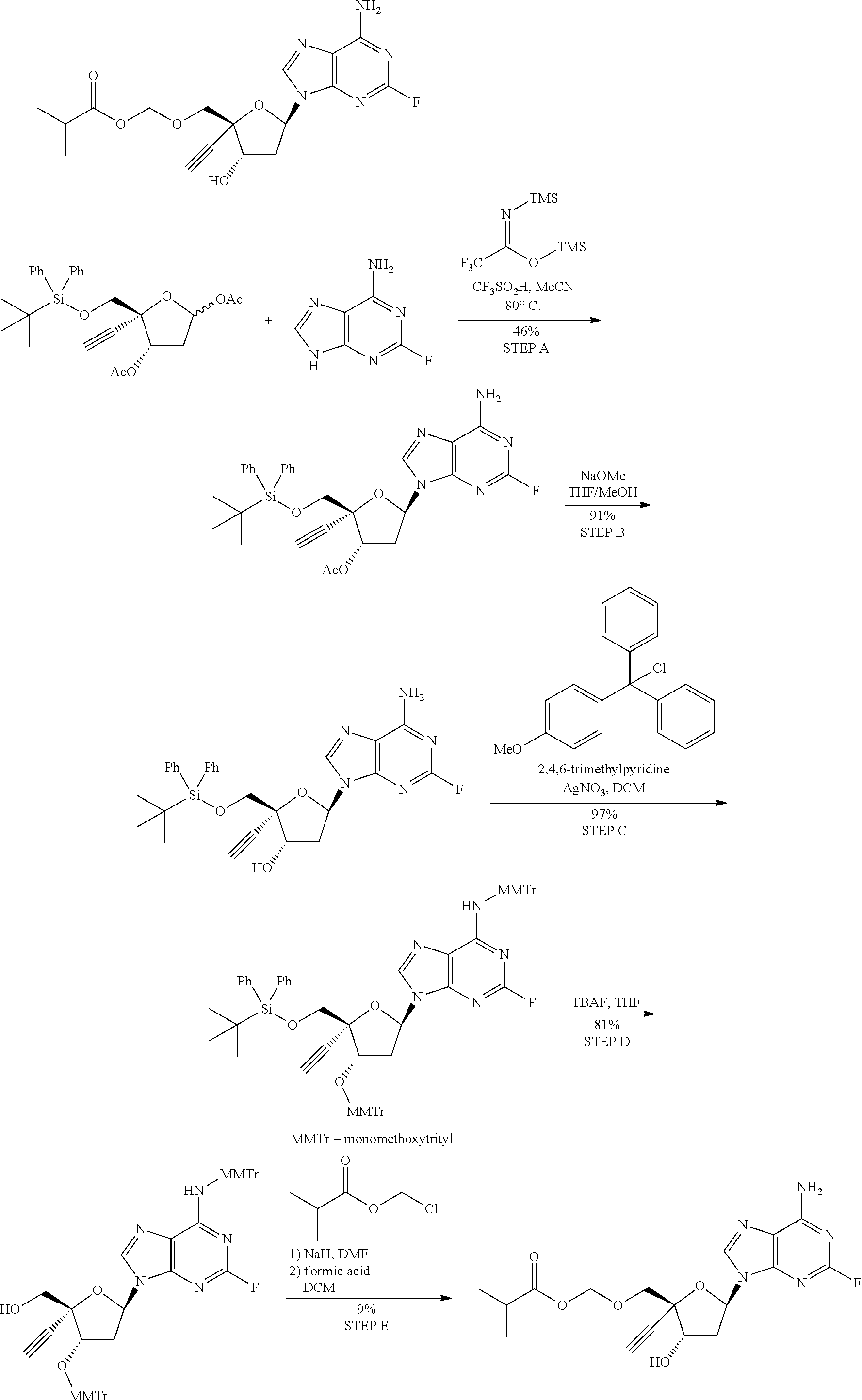

Step A: (2R,3S,5R)-5-(6-amino-2-fluoro-9H-purin-9-yl)-2-(((tert-butyldiphenylsilyl)oxy)methyl)-2-ethynyltetrahydrofuran-3-yl acetate. A suspension of 2-fluoro-9H-purin-6-amine (0.545 g, 3.56 mmol) in anhydrous MeCN (10 mL) in a screw-capped glass pressure vessel under a nitrogen atmosphere was treated with trimethylsilyl 2,2,2-trifluoro-N-(trimethylsilyl)acetimidate (1.89 ml, 7.12 mmol) and heated to 80° C. with stirring in an oil bath. After 45 minutes most of the solid had dissolved. The solution was treated with (4S,5R)-5-(((tert-butyldiphenylsilyl)oxy)methyl)-5-ethynyltetrahydrofuran-2,4-diyl diacetate (1.14 g, 2.37 mmol, prepared according to Org. Lett., Vol. 13, No. 19, 2011) dissolved in MeCN (9 mL) followed by freshly prepared 0.2M trifluoromethanesulfonic acid/MeCN (2.37 ml, 0.474 mmol) (prepared by dissolving 44 μL of triflic acid in 2.5 mL of MeCN). The temperature was maintained at 80° C. After 1.5 hour at 80° C. LCMS indicated complete reaction. The solution was cooled to RT, quenched by addition of 1M aqueous HCl (3 mL). After stirring the mixture briefly, it was partitioned between saturated aqueous NaHCO₃ and EtOAc and the phases separated. The aqueous phase was extracted with EtOAc (2×). The combined EtOAc solutions were dried over Na₂SO₄ and concentrated at reduced pressure to give a tan solid. This material was subjected to flash chromatography (silica gel, 0-100% EtOAc/DCM) and the higher R_{f} component isolated to afford the title compound (0.63 g, 46%) as a white solid. LCMS (ESI) m/z calcd for C₃₀H₃₂FN₅O₄Si: 573.2. Found: 574.4 (M+1)⁺. ¹H NMR (400 MHz, METHANOL-d₄) δ 8.10 (s, 1H), 7.59-7.67 (m, 4H), 7.26-7.45 (m, 6H), 6.39 (t, J=6.6 Hz, 1H), 5.91 (dd, J=7.0, 5.5 Hz, 1H), 3.97 (d, J=10.9 Hz, 1H), 3.86 (d, J=10.9 Hz, 1H), 3.05-3.18 (m, 2H), 2.64-2.74 (m, 1H), 2.14 (s, 3H), 0.97-1.04 (m, 9H).

Step B: (2R,3S,5R)-5-(6-amino-2-fluoro-9H-purin-9-yl)-2-(((tert-butyldiphenylsilyl)oxy)methyl)-2-ethynyltetrahydrofuran-3-ol. To a stirred solution of (2R,3S,5R)-5-(6-amino-2-fluoro-9H-purin-9-yl)-2-(((tert-butyldiphenylsilyl)oxy)methyl)-2-ethynyltetrahydrofuran-3-yl acetate (0.62 g, 1.08 mmol) in 1:1 THF/MeOH (4 mL) was added 25% NaOMe/MeOH (3 drops). The resulting solution was stirred at RT. After 30 minutes LCMS indicated complete reaction. The solution was treated with glacial AcOH (5 drops) and concentrated to dryness at reduced pressure. The residue was partitioned between 8:2 chloroform/iPrOH and half-saturated aqueous NaHCO₃ and the phases separated. The aqueous phase was extracted with two additional portions of 8:2 chloroform/iPrOH. The combined organic solutions were dried over Na₂SO₄ and concentrated to dryness at reduced pressure to afford the title compound (0.52 g, 91%) as a white solid. LCMS (ESI) m/z calcd for C₂₈H₃₀FN₅O₃Si: 531.2. Found: 532.3 (M+1)⁺.¹H NMR (400 MHz, METHANOL-d₄) δ 8.17 (s, 1H), 7.53-7.66 (m, 4H), 7.22-7.45 (m, 6H), 6.32 (dd, J=7.8, 3.1 Hz, 1H), 5.01 (t, J=7.8 Hz, 1H), 3.87 (q, J=11.3 Hz, 2H), 3.05 (s, 1H), 2.90-2.99 (m, 1H), 2.63-2.72 (m, 1H), 0.94 (s, 9H).

Step C: 9-((2R,4S,5R)-5-(((tert-butyldiphenylsilyl)oxy)methyl)-5-ethynyl-4-((4-methoxyphenyl)diphenylmethoxy)tetrahydrofuran-2-yl)-2-fluoro-N-((4-methoxyphenyl)diphenylmethyl)-9H-purin-6-amine. To a stirred suspension of (2R,3S,5R)-5-(6-amino-2-fluoro-9H-purin-9-yl)-2-(((tert-butyldiphenylsilyl)oxy)methyl)-2-ethynyltetrahydrofuran-3-ol (0.510 g, 0.959 mmol) in DCM (8 mL) was added silver nitrate (0.489 g, 2.88 mmol), 2,4,6-trimethylpyridine (0.766 ml, 5.76 mmol), and (chloro(4-methoxyphenyl)methylene)dibenzene (0.889 g, 2.88 mmol). The resulting orange suspension was stirred at RT. After 2 hours LCMS indicated complete reaction. The mixture was diluted with EtOAc and filtered through celite to remove solids. The filtrate was washed with 10% aqueous citric acid (2×), saturated aqueous NaHCO₃ (2×), dried over Na₂SO₄ and concentrated at reduced pressure to give a pale yellow foam. This material was subjected to flash chromatography (silica gel, 0-100% EtOAc/hexanes) to afford the title compound (1.00 g, 97%) as a white foam. LCMS (ESI) m/z calcd for C₆₈H₆₂FN₅O₅Si: 1075.5. Found: 1076.7 (M+1)⁺. ¹H NMR (400 MHz, CDCl₃) δ 7.12-7.62 (m, 35H), 6.98 (s, 1H), 6.74-6.82 (m, 4H), 6.22 (t, J=6.6 Hz, 1H), 4.75 (t, J=5.9 Hz, 1H), 3.93 (d, J=11.3 Hz, 1H), 3.86 (d, J=11.3 Hz, 1H), 3.77 (s, 3H), 3.75 (s, 3H), 2.77 (s, 1H), 1.71 (t, J=6.3 Hz, 2H), 0.87 (s, 9H).

Step D: ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methanol. To a stirred solution of 9-((2R,4S,5R)-5-(((tert-butyldiphenylsilyl)oxy)methyl)-5-ethynyl-4-((4-methoxyphenyl)diphenylmethoxy)tetrahydrofuran-2-yl)-2-fluoro-N-((4-methoxyphenyl)diphenylmethyl)-9H-purin-6-amine (0.99 g, 0.92 mmol) in THF (8 mL) was added 1M TBAF/THF (1.38 ml, 1.38 mmol) by dropwise addition. The resulting solution was stirred at RT. After 1 hour LCMS indicated complete reaction. The solution was treated with glacial AcOH (0.10 mL) and concentrated at reduced pressure. The residue was dissolved in MeOH/DCM and again concentrated to dryness. The residue was subjected to flash chromatography (silica gel, 0-100% EtOAc/hexanes) to afford the title compound (0.623 g, 81%) as a white solid. LCMS (ESI) m/z calcd for C₅₂H₄₄FN₅O₅: 837.3. Found: 838.6 (M+1)⁺. ¹H NMR (400 MHz, DMSO-d₆) δ 8.04 (s, 1H), 8.00 (s, 1H), 7.47-7.54 m, 4H), 7.12-7.38 (m, 20H), 6.79-6.88 (m, 4H), 6.04 (t, J=6.3 Hz, 1H), 5.15 (t, J=6.1 Hz, 1H), 4.47 (t, J=6.1 Hz, 1H), 3.84 (s, 1H), 3.69 (s, 3H), 3.67 (s, 3H), 3.49-3.57 (m, 1H), 3.38-3.47 (m, 1H), 1.63-1.72 (m, 1H), 1.49-1.58 (m, 1H).

Step E: (((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)methyl isobutyrate. To a stirred solution of ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methanol (32 mg, 0.038 mmol) in DMF (0.3 mL) at 0° C. was added sodium hydride, 60% dispersion in mineral oil, (3.05 mg, 0.076 mmol) and the mixture was stirred at ambient temperature for 30 minutes. A solution of chloromethyl isobutyrate (5.74 mg, 0.042 mmol) in DMF (0.1 mL) was added and the mixture was stirred at ambient temperature for one hour. The mixture was partitioned between EtOAc and saturated NaHCO₃ and the phases were separated. The organic phase was washed with water, then brine, dried over Na₂SO₄, filtered and concentrated. The residue was purified on silica gel (4 g column, 0-30% DCM/EtOAc) to afford a mixture of starting material and desired 5′-alkylated product as a white solid. DCM (1.0 mL) was added followed by formic acid (1 mL, 26.1 mmol) and the orange mixture was allowed to stir at ambient temperature. After 30 minutes the mixture was concentrated and then purified by RP-HPLC to afford the title compound (1.4 mg, 9%) as a colorless residue. LCMS (ESI) m/z calcd for C₁₇H₂₀FN₅O₅: 393.1. Found: 394.2 (M+1)⁺. ¹H NMR (400 MHz, METHANOL-d₄) δ 8.19 (s, 1H), 6.33 (dd, J=4.3, 7.4 Hz, 1H), 5.28 (d, J=6.3, 1H), 5.24 (d, J=6.3, 1H), 4.77-4.71 (m, 1H), 3.98 (d, J=10.9, 1H), 3.91 (d, J=10.9, 1H), 3.14 (s, 1H), 2.81-2.72 (m, 1H), 2.67-2.46 (m, 2H), 1.13-1.09 (m, 6H).

### Example 2

### (((2R,3S,5R)-5-(6-amino-2-fluoro-9H-purin-9-yl)-2-ethynyl-3-hydroxytetrahydrofuran-2-yl)methoxy)methyl isopropyl carbonate

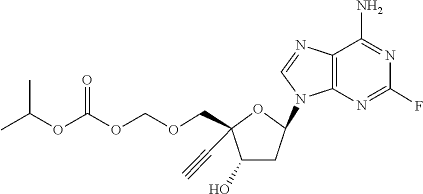 [SMILES: C#C[C@]1(COCOC(=O)OC(C)C)O[C@@H](n2cnc3c(N)nc(F)nc32)C[C@@H]1O]

The title compound was made in a similar manner as Example 1 except using chloromethyl isopropyl carbonate in Step E. LCMS (ESI) m/z calcd for C₁₇H₂₀FN₅O₆: 409.1. Found: 410.3 (M+1)⁺. ¹H NMR (400 MHz, METHANOL-d₄) δ 8.21 (s, 1H), 6.34 (dd, J=4.1, 6.8 Hz, 1H), 5.31-5.21 (m, 2H), 4.91-4.78 (m, 1H, overlapping water peak), 4.78-4.71 (m, 1H), 4.05-3.91 (m, 2H), 3.14 (s, 1H), 2.82-2.71 (m, 1H), 2.69-2.57 (m, 1H), 1.27-1.20 (m, 6H).

### Example 3

### ethyl (9-((2R,4S,5R)-5-ethynyl-4-hydroxy-5-(hydroxymethyl)tetrahydrofuran-2-yl)-2-fluoro-9H-purin-6-yl)carbamate

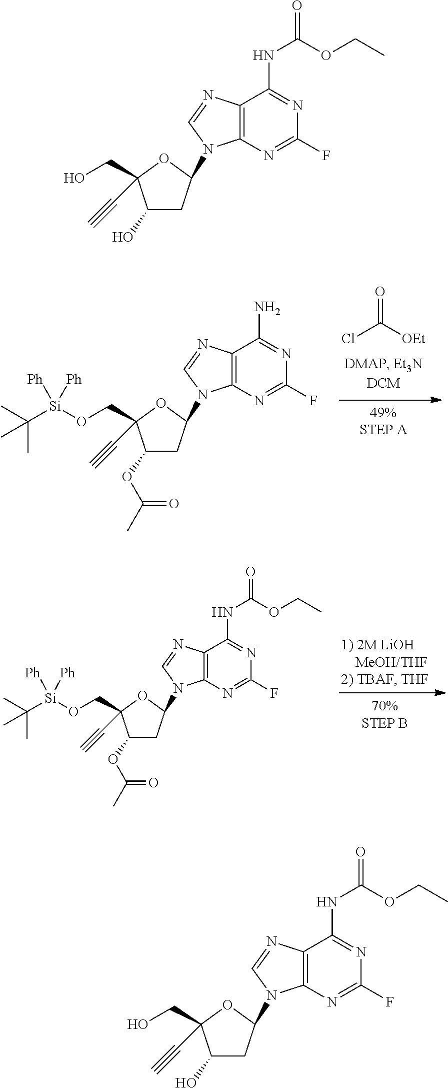

Step A: (2R,3S,5R)-2-(((tert-butyldiphenylsilyl)oxy)methyl)-5-(6-((ethoxycarbonyl)amino)-2-fluoro-9H-purin-9-yl)-2-ethynyltetrahydrofuran-3-yl acetate. To a solution of (2R,3S,5R)-5-(6-amino-2-fluoro-9H-purin-9-yl)-2-(((tert-butyldiphenylsilyl)oxy)methyl)-2-ethynyltetrahydrofuran-3-yl acetate (32 mg, 0.056 mmol), DMAP (6.81 mg, 0.056 mmol) and triethylamine (0.016 mL, 0.112 mmol) in DCM (0.4 mL) at 0° C. was added a solution of ethyl chloroformate (6.96 μl, 0.073 mmol) in DCM (93 uL) and the mixture was stirred at ambient temperature for 20 minutes. The mixture was cooled to 0° C. and additional triethylamine (0.016 mL, 0.112 mmol) and ethyl chloroformate (6.96 μl, 0.073 mmol) in DCM (93 ul) were added. The mixture was stirred at ambient temperature for 30 minutes. The mixture was diluted with DCM, washed with saturated NaHCO₃, then brine, dried over Na₂SO₄, filtered and concentrated. The residue was purified on silica gel (4 g column, 0-50% DCM/EtOAc) to afford the title compound (17.5 mg, 49%) as a colorless residue. LCMS (ESI) m/z calcd for C₃₃H₃₆FN₅O₆Si: 645.2. Found: 646.4 (M+1)⁺. ¹H NMR (400 MHz, CDCl₃) δ 8.10 (s, 1H), 8.08 (s, 1H), 7.69-7.64 (m, 4H), 7.48-7.42 (m, 2H), 7.41-7.35 (m, 4H), 6.54 (dd, J=5.9, 7.4 Hz, 1H), 5.82 (dd, J=3.9, 7.0 Hz, 1H), 4.35 (q, J=7.3 Hz, 2H), 4.06 (d, J=11.3 Hz, 1H), 3.96 (d, J=11.3 Hz, 1H), 2.91-2.83 (m, 1H), 2.75-2.67 (m, 1H), 2.59 (s, 1H), 2.17 (s, 3H), 1.37 (t, J=7.0 Hz, 3H), 1.11-1.07 (m, 9H).

Step B: ethyl(9-((2R,4S,5R)-5-ethynyl-4-hydroxy-5-(hydroxymethyl)tetrahydrofuran-2-yl)-2-fluoro-9H-purin-6-yl)carbamate. To a stirred solution of (2R,3S,5R)-2-(((tert-butyldiphenylsilyl)oxy)methyl)-5-(6-((ethoxycarbonyl)amino)-2-fluoro-9H-purin-9-yl)-2-ethynyltetrahydrofuran-3-yl acetate (17.5 mg, 0.027 mmol) in THF (0.3 mL) and Methanol (0.1 mL) was added 2M LiOH (0.027 mL, 0.054 mmol) at ambient temperature and the mixture was allowed to stir for 35 minutes. 1N HCl (100 uL) was added, then diluted with water and the mixture was extracted with EtOAc. The extracts were washed with brine, dried over Na₂SO₄, filtered and concentrated to a colorless residue. The residue was dissolved in THF (0.400 mL), treated with TBAF (0.032 mL, 0.032 mmol) at ambient temperature and the mixture was allowed to stir for 20 minutes. AcOH (6 drops) was added, the mixture was stirred for several minutes and then concentrated. The residue was purified on silica gel (4 g column, 0-10% DCM/MeOH) to afford the title compound (7 mg, 70%) as a white residue. LCMS (ESI) m/z calcd for C₁₅H₁₆FN₅O₅: 365.1. Found: 366.2 (M+1)⁺. ¹H NMR (400 MHz, METHANOL-d₄) δ 8.49 (s, 1H), 6.43 (dd, J=4.3, 7.4 Hz, 1H), 4.82-4.75 (m, 1H), 4.31 (q, J=7.0 Hz, 2H), 3.86 (d, J=12.1 Hz, 1H), 3.77 (d, J=12.5 Hz, 1H), 3.11 (s, 1H), 2.86-2.77 (m, 1H), 2.71-2.62 (m, 1H), 1.36 (t, J=7.0 Hz, 3H).

### Example 4

### ((2R,3S,5R)-5-(6-amino-2-fluoro-9H-purin-9-yl)-2-ethynyl-3-hydroxytetrahydrofuran-2-yl)methyl dodecyl carbonate

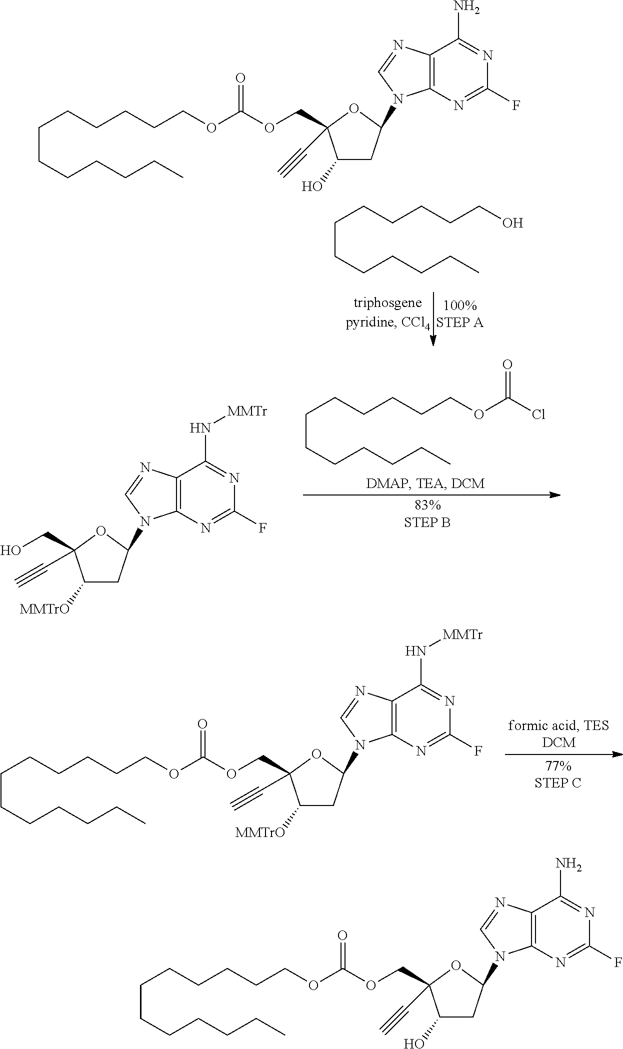

Step A: dodecyl carbonochloridate. To a stirred solution of dodecan-1-ol (373 mg, 2.0 mmol) and pyridine (0.178 mL, 2.200 mmol) in CCl₄ (20 mL) cooled in an ice-salt mixture was added triphosgene (593 mg, 2.000 mmol). The mixture was stirred for several minutes at −15° C. and then warmed to ambient temperature and stirred overnight. The mixture was diluted with ether, then filtered over Celite. The filtrate was concentrated to afford dodecyl carbonochloridate (531 mg, quantitative yield) as a colorless oil. ¹H NMR (400 MHz, CDCl₃) δ=4.32 (t, J=6.6 Hz, 2H), 1.81-1.65 (m, 2H), 1.45-1.22 (m, 18H), 0.94-0.82 (m, 3H).

Step B: dodecyl (((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl) carbonate. To a stirred solution of ((2R,3S,5R)-2-ethynyl-5-(2-fluoro-6-(((4-methoxyphenyl)diphenylmethyl)amino)-9H-purin-9-yl)-3-((4-methoxphenyl)diphenylmethoxy)tetrahydrofuran-2-yl)methanol (317 mg, 0.378 mmol) and DMAP (46.2 mg, 0.378 mmol) in DCM (2.5 mL) at 0° C. was added triethylamine (0.158 mL, 1.135 mmol), followed by a solution of dodecyl carbonochloridate (226 mg, 0.908 mmol) in DCM (1.0 mL). The mixture was stirred at 0° C. for 5 minutes and then warmed to ambient temperature and stirred for 4 hours. The mixture was concentrated and then purified on silica gel (24 g gold column, 0-50% DCM/EtOAc) to afford the title compound (330 mg, 83%) as a white solid. LCMS (ESI) m/z calcd for C₆₅H₆₈FN₅O₇: 1049.5. Found: 1050.7 (M+1)⁺. ¹H NMR (400 MHz, CDCl₃) δ 7.63 (s, 1H), 7.57-7.52 (m, 4H), 7.43-7.38 (m, 2H), 7.34-7.19 (m, 18H), 7.02 (s, 1H), 6.84-6.77 (m, 4H), 6.16 (dd, J=4.1, 7.6 Hz, 1H), 4.62-4.57 (m, 1H), 4.31 (d, J=11.7 Hz, 1H), 4.06-4.01 (m, 2H), 3.97 (d, J=11.7 Hz, 1H), 3.79 (s, 3H), 3.76 (s, 3H), 2.85 (s, 1H), 2.21-2.12 (m, 1H), 1.84-1.76 (m, 1H), 1.65-1.57 (m, 2H), 1.35-1.20 (m, 18H), 0.92-0.85 (m, 3H).

Step C: ((2R,3S,5R)-5-(6-amino-2-fluoro-9H-purin-9-yl)-2-ethynyl-3-hydroxytetrahydrofuran-2-yl)methyl dodecyl carbonate. To a stirred solution of dodecyl (((2R,3S,5R)-2-ethynyl-5-(2-fluoro-6-(((4-methoxphenyl)diphenylmethyl)amino)-9H-purin-9-yl)-3-((4-methoxyphenyl)diphenylmethoxy)tetrahydrofuran-2-yl)methyl) carbonate (464 mg, 0.442 mmol) in DCM (5.0 mL) at ambient temperature was slowly added formic acid (1.0 mL, 26.1 mmol) and the mixture was allowed to stir for 4.5 hours. Triethylsilane (0.141 mL, 0.884 mmol) was added and the mixture was stirred at ambient temperature for one hour. The mixture was concentrated and then co-concentrated with EtOH to afford a white solid. The material was purified on silica gel (24 g gold column, 0-10% DCM/MeOH) to afford the title compound (171 mg, 77%) as a white solid. LCMS (ESI) m/z calcd for C₂₅H₃₆FN₅O₅: 505.3. Found: 506.4 (M+1)⁺. ¹H NMR (400 MHz, DMSO-d₆) δ 8.25 (s, 1H), 7.99-7.73 (m, 2H), 6.25 (dd, J=4.3, 7.8 Hz, 1H), 5.84 (d, J=5.5 Hz, 1H), 4.71-4.59 (m, 1H), 4.42 (d, J=11.3 Hz, 1H), 4.19 (d, J=11.7 Hz, 1H), 4.05-3.88 (m, 2H), 3.65 (s, 1H), 2.82-2.73 (m, 1H), 2.54-2.41 (m, 1H, overlapping solvent peak), 1.56-1.45 (m, 2H), 1.33-1.11 (m, 18H), 0.84 (t, J=6.6 Hz, 3H).

### Example 5

### ((2R,3S,5R)-5-(6-amino-2-fluoro-9H-purin-9-yl)-2-ethynyl-3-hydroxytetrahydrofuran-2-yl)methyl ethyl carbonate

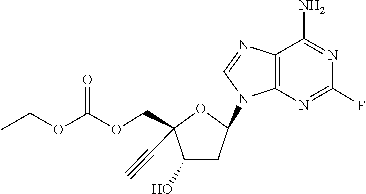 [SMILES: C#C[C@]1(COC(=O)OCC)O[C@@H](n2cnc3c(N)nc(F)nc32)C[C@@H]1O]

The title compound was made in a similar manner as Example 4 except using ethyl carbonochloridate in Step A. LCMS (ESI) m/z calcd for C₁₅H₁₆FN₅O₅: 365.1. Found: 366.1 (M+1)⁺. ¹H NMR (400 MHz, DMSO-d₆) δ 8.25 (s, 1H), 8.01-7.71 (m, 2H), 6.25 (dd, J=4.3, 7.8 Hz, 1H), 5.84 (d, J=5.5 Hz, 1H), 4.71-4.61 (m, 1H), 4.42 (d, J=11.3 Hz, 1H), 4.20 (d, J=11.7 Hz, 1H), 4.08-3.99 (m, 2H), 3.66 (s, 1H), 2.84-2.73 (m, 1H), 2.56-2.39 (m, 1H, overlapping DMSO peak), 1.14 (t, J=7.2 Hz, 3H).

### Example 6

### (((2R,3S,5R)-5-(6-amino-2-fluoro-9H-purin-9-yl)-2-ethynyl-3-hydroxytetrahydrofuran-2-yl)methoxy)methyl decyl carbonate

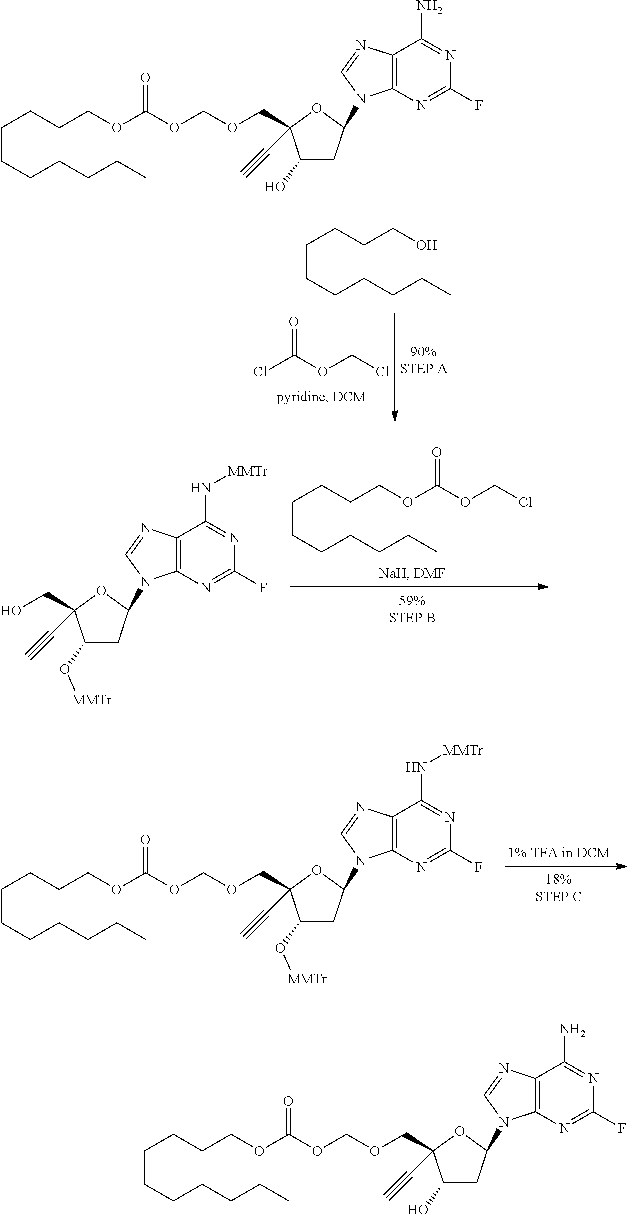

Step A: Chloromethyl decyl carbonate. To a stirred solution of chloromethyl carbonochloridate (0.690 mL, 7.76 mmol) in DCM (30 mL) at 0° C. was added dropwise over one hour (via syringe pump) a solution of decan-1-ol (1.481 mL, 7.76 mmol) and pyridine (0.659 mL, 8.14 mmol) in DCM (8 mL). The mixture was stirred at 0° C. for 30 minutes. The mixture was diluted with DCM, washed with 0.5M HCl, water, saturated NaHCO₃, and finally brine. The organic phase was dried over Na₂SO₄, filtered and concentrated. The residue was purified on silica gel (24 g column, 0-30% hexanes/EtOAc) to afford the title compound (1.76 g, 90%) as a colorless oil. ¹H NMR (400 MHz, CDCl₃) δ 5.74 (s, 2H), 4.23 (t, J=6.6 Hz, 2H), 1.75-1.66 (m, 2H), 1.44-1.16 (m, 14H), 0.89 (t, J=6.6 Hz, 3H).

Step B: decyl ((((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)methyl) carbonate. To a stirred solution of ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methanol (31 mg, 0.037 mmol) in DMF (0.5 mL) at 0° C. was added sodium hydride, 60% dispersion in mineral oil (2.96 mg, 0.074 mmol). The mixture was allowed to stir at ambient temperature for 10 minutes. A solution of chloromethyl decyl carbonate (27.8 mg, 0.111 mmol) in DMF (150 uL) was added and the mixture was stirred for 10 minutes. Saturated NHCl₄ was added and the mixture was stirred for several minutes. The mixture was diluted with EtOAc, washed with water, then brine, dried over Na₂SO₄, filtered and concentrated. The residue was purified on silica gel (4 g column, 0-50% DCM/EtOAc) to afford the title compound (23 mg, 59%) as a colorless residue. LCMS (ESI) m/z calcd for C₁₅H₁₆FN₅O₅: 1051.5. Found: 1052.5 (M+1)⁺.

Step C: (((2R,3S,5R)-5-(6-amino-2-fluoro-9H-purin-9-yl)-2-ethynyl-3-hydroxytetrahydrofuran-2-yl)methoxy)methyl decyl carbonate. To decyl ((((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)methyl) carbonate (23 mg, 0.022 mmol) was added 1% TFA in DCM (0.8 ml) and the mixture was allowed to stir at ambient temperature for one hour. The mixture was concentrated and then purified by RP-HPLC (C18, 10-100% MeCN/water with 0.1% FA) to afford the title compound (2.0 mg, 18%) as a white solid. LCMS (ESI) m/z calcd for C₂₄H₃₄FN₅O₆: 507.3. Found: 508.3 (M+1)⁺. ¹H NMR (400 MHz, METHANOL-d₄) δ 8.21 (s, 1H), 6.33 (dd, J=4.3, 7.4 Hz, 1H), 5.29 (d, J=6.6 Hz, 1H), 5.24 (d, J=6.6 Hz, 1H), 4.76-4.70 (m, 1H), 4.12-4.05 (m, 2H), 4.02 (d, J=11.3 Hz, 1H), 3.95 (d, J=11.3 Hz, 1H), 3.14 (s, 1H), 2.78-2.69 (m, 1H), 2.67-2.56 (m, 1H), 1.63-1.54 (m, 2H), 1.36-1.19 (m, 14H), 0.88 (t, J=6.8 Hz, 3H).

### Example 7

### ethyl (9-((2R,4S,5R)-4-((ethoxycarbonyl)oxy)-5-ethynyl-5-(hydroxymethyl)tetrahydrofuran-2-yl)-2-fluoro-9H-purin-6-yl)carbamate

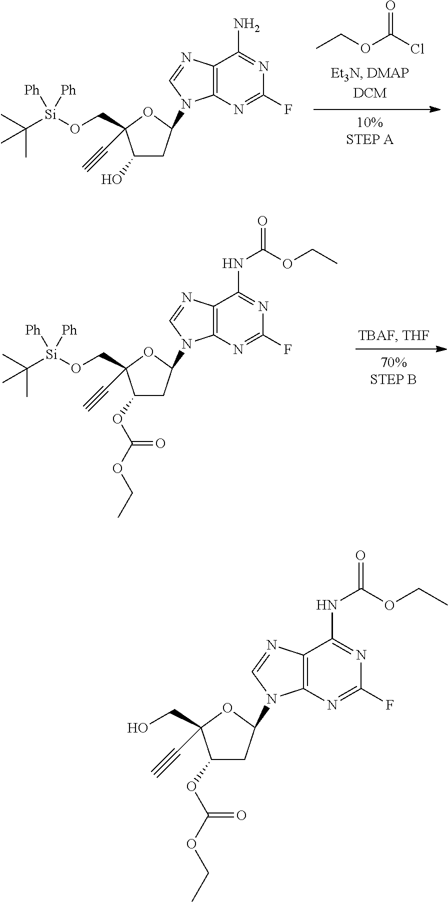

Step A: ethyl (9-((2R,4S,5R)-5-(((tert-butyldiphenylsilyl)oxy)methyl)-4-((ethoxycarbonyl)oxy)-5-ethynyltetrahydrofuran-2-yl)-2-fluoro-9H-purin-6-yl)carbamate. To a stirred solution of (2R,3S,5R)-5-(6-amino-2-fluoro-9H-purin-9-yl)-2-(((tert-butyldiphenylsilyl)oxy)methyl)-2-ethynyltetrahydrofuran-3-ol (50 mg, 0.094 mmol), triethylamine (0.066 mL, 0.470 mmol) and DMAP (11.49 mg, 0.094 mmol) in DCM (0.8 mL) at 0° C. was added ethyl carbonochloridate (0.018 mL, 0.188 mmol) in DCM (82 uL). The mixture was stirred at 0° C. for 5 minutes, then warmed to ambient temperature and stirred for 90 minutes. The mixture was diluted with EtOAc, washed with saturated NaHCO₃ mixed with brine, followed by brine and finally dried (Na₂SO₄), filtered and concentrated. The residue was purified on silica gel (4 g gold column, 0-50% DCM/EtOAc) to afford the title compound (6.4 mg, 10%) as a colorless residue. LCMS (ESI) m/z calcd for C₃₄H₃₈FN₅O₇Si: 675.3. Found: 676.4 (M+1)⁺. ¹H NMR (400 MHz, CDCl₃) δ 8.10 (s, 1H), 8.08 (s, 1H), 7.68-7.62 (m, 4H), 7.47-7.41 (m, 2H), 7.40-7.34 (m, 4H), 6.54-6.49 (m, 1H), 5.71 (dd, J=4.3, 6.6 Hz, 1H), 4.35 (q, J=7.3 Hz, 2H), 4.31-4.24 (m, 2H), 4.08 (d, J=11.3 Hz, 1H), 3.98 (d, J=10.9 Hz, 1H), 3.00-2.92 (m, 1H), 2.84-2.76 (m, 1H), 2.63 (s, 1H), 1.39-1.34 (m, 6H), 1.08 (s, 9H).

Step B: ethyl (9-((2R,4S,5R)-4-((ethoxycarbonyl)oxy)-5-ethynyl-5-(hydroxymethyl)tetrahydrofuran-2-yl)-2-fluoro-9H-purin-6-yl)carbamate. To a stirred solution of ethyl (9-((2R,4S,5R)-5-(((tert-butyldiphenylsilyl)oxy)methyl)-4-((ethoxycarbonyl)oxy)-5-ethynyltetrahydrofuran-2-yl)-2-fluoro-9H-purin-6-yl)carbamate (6.4 mg, 9.47 μmol) in THF (0.3 mL) at ambient temperature was added TBAF, 1M solution in THF (0.012 mL, 0.012 mmol) and the mixture was allowed to stir at ambient temperature for 10 minutes. AcOH (5 drops) was added and the mixture was concentrated to dryness. The residue was purified by RP-HPLC to afford the title compound (2.9 mg, 70%) as a white solid. LCMS (ESI) m/z calcd for C₁₈H₂₀FN₅O₇: 437.1. Found: 438.2 (M+1)⁺. ¹H NMR (400 MHz, DMSO-d₆) δ 11.29-10.76 (m, 1H), 8.60 (s, 1H), 6.42-6.37 (m, 1H), 5.63-5.53 (m, 1H), 5.48 (dd, J=3.7, 6.4 Hz, 1H), 4.23-4.14 (m, 4H), 3.74-3.59 (m, 3H), 3.15-3.06 (m, 1H), 2.71-2.62 (m, 1H), 1.28-1.22 (m, 6H).

### Example 8

### (2R,3S,5R)-5-(6-amino-2-fluoro-9H-purin-9-yl)-2-ethynyl-2-(hydroxymethyl)tetrahydrofuran-3-yl ethyl carbonate

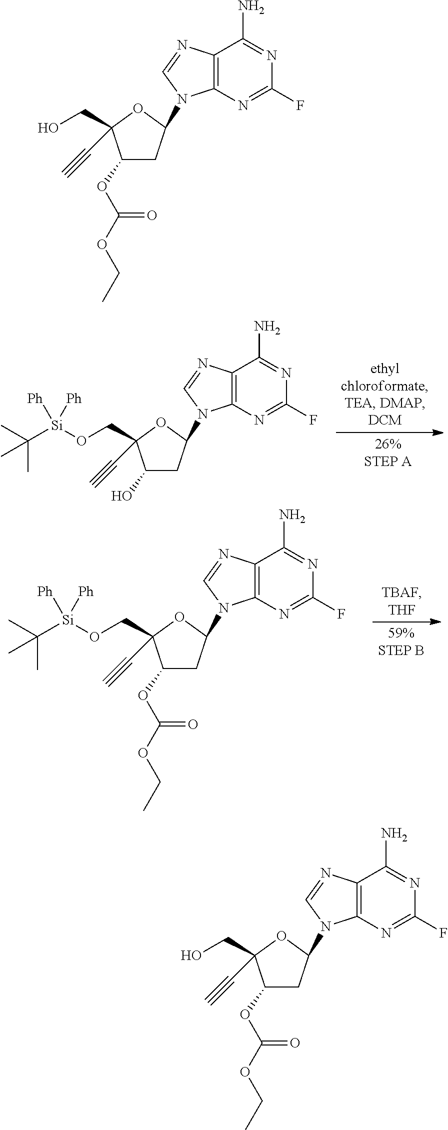 [SMILES: C#C[C@]1(CO)O[C@@H](n2cnc3c(N)nc(F)nc32)C[C@@H]1OC(=O)OCC ; C#C[C@]1(CO[Si](c2ccccc2)(c2ccccc2)C(C)(C)C)O[C@@H](n2cnc3c(N)nc(F)nc32)C[C@@H]1O ; C#C[C@]1(CO[Si](c2ccccc2)(c2ccccc2)C(C)(C)C)O[C@@H](n2cnc3c(N)nc(F)nc32)C[C@@H]1OC(=O)OCC ; C#C[C@]1(CO)O[C@@H](n2cnc3c(N)nc(F)nc32)C[C@@H]1OC(=O)OCC]

Step A: (2R,3S,5R)-5-(6-amino-2-fluoro-9H-purin-9-yl)-2-(((tert-butyldiphenylsilyl)oxy)methyl)-2-ethynyltetrahydrofuran-3-yl ethyl carbonate. To a stirred solution of (2R,3S,5R)-5-(6-amino-2-fluoro-9H-purin-9-yl)-2-(((tert-butyldiphenylsilyl)oxy)methyl)-2-ethynyltetrahydrofuran-3-ol (178 mg, 0.335 mmol), TEA (0.233 mL, 1.674 mmol) and DMAP (40.9 mg, 0.335 mmol) in DCM (3.0 mL) at 0° C. was added ethyl chloroformate (0.064 mL, 0.670 mmol) in DCM (0.44 mL). The mixture was stirred at 0° C. for 5 minutes, then stirred at ambient temperature for 2 hours. The mixture was diluted with EtOAc, washed with saturated aqueous NaHCO₃ mixed with brine, followed by brine only and finally dried (Na₂SO₄), filtered and concentrated. The residue was purified on silica gel (0-50% EtOAc/DCM) to afford the title compound (52 mg, 26%) as a white solid. LCMS (ESI) m/z calcd for C₃₁H₃₄FN₅O₅Si: 603.2. Found: 604.4 (M+1)⁺. ¹H NMR (400 MHz, CDCl₃) δ 7.94 (s, 1H), 7.71-7.63 (m, 4H), 7.47-7.41 (m, 2H), 7.40-7.34 (m, 4H), 6.50-6.45 (m, 1H), 5.89 (br s, 2H), 5.72 (dd, J=4.4, 6.8 Hz, 1H), 4.32-4.24 (m, 2H), 4.08 (d, 11.2 Hz, 1H), 3.98 (d, 11.2 Hz, 1H), 3.00-2.92 (m, 1H), 2.81-2.74 (m, 1H), 2.61 (s, 1H), 1.36 (t, J=7.2 Hz, 3H), 1.13-1.05 (m, 9H).

Step B: (2R,3S,5R)-5-(6-amino-2-fluoro-9H-purin-9-yl)-2-ethynyl-2-(hydroxymethyl)tetrahydrofuran-3-yl ethyl carbonate. To a stirred solution of (2R,3S,5R)-5-(6-amino-2-fluoro-9H-purin-9-yl)-2-(((tert-butyldiphenylsilyl)oxy)methyl)-2-ethynyltetrahydrofuran-3-yl ethyl carbonate (52 mg, 0.086 mmol) in THF (1.2 mL) at ambient temperature was added TBAF, 1M solution in THF (0.129 mL, 0.129 mmol) and the mixture was allowed to stir for 20 minutes. The mixture was concentrated and then purified on silica gel (0-10% MeOH/DCM) to afford the title compound (19 mg, 59%) as a white solid. LCMS (ESI) m/z calcd for C₁₅H₁₆FN₅O₅: 365.1. Found: 366.2 (M+1)⁺. ¹H NMR (400 MHz, DMSO-d₆) δ 8.32 (s, 1H), 8.02-7.74 (m, 2H), 6.31 (dd, J=6.3, 8.0 Hz, 1H), 5.56 (dd, J=5.6, 7.0 Hz, 1H), 5.44 (dd, J=3.2, 6.6 Hz, 1H), 4.23-4.13 (m, 2H), 3.75-3.58 (m, 3H), 3.08-2.99 (m, 1H), 2.64-2.57 (m, 1H), 1.25 (t, J=7.2 Hz, 3H).

### Example 9

### (2R,3S,5R)-5-(6-amino-2-fluoro-9H-purin-9-yl)-2-ethynyl-2-(hydroxymethyl)tetrahydrofuran-3-yl nonadecyl carbonate

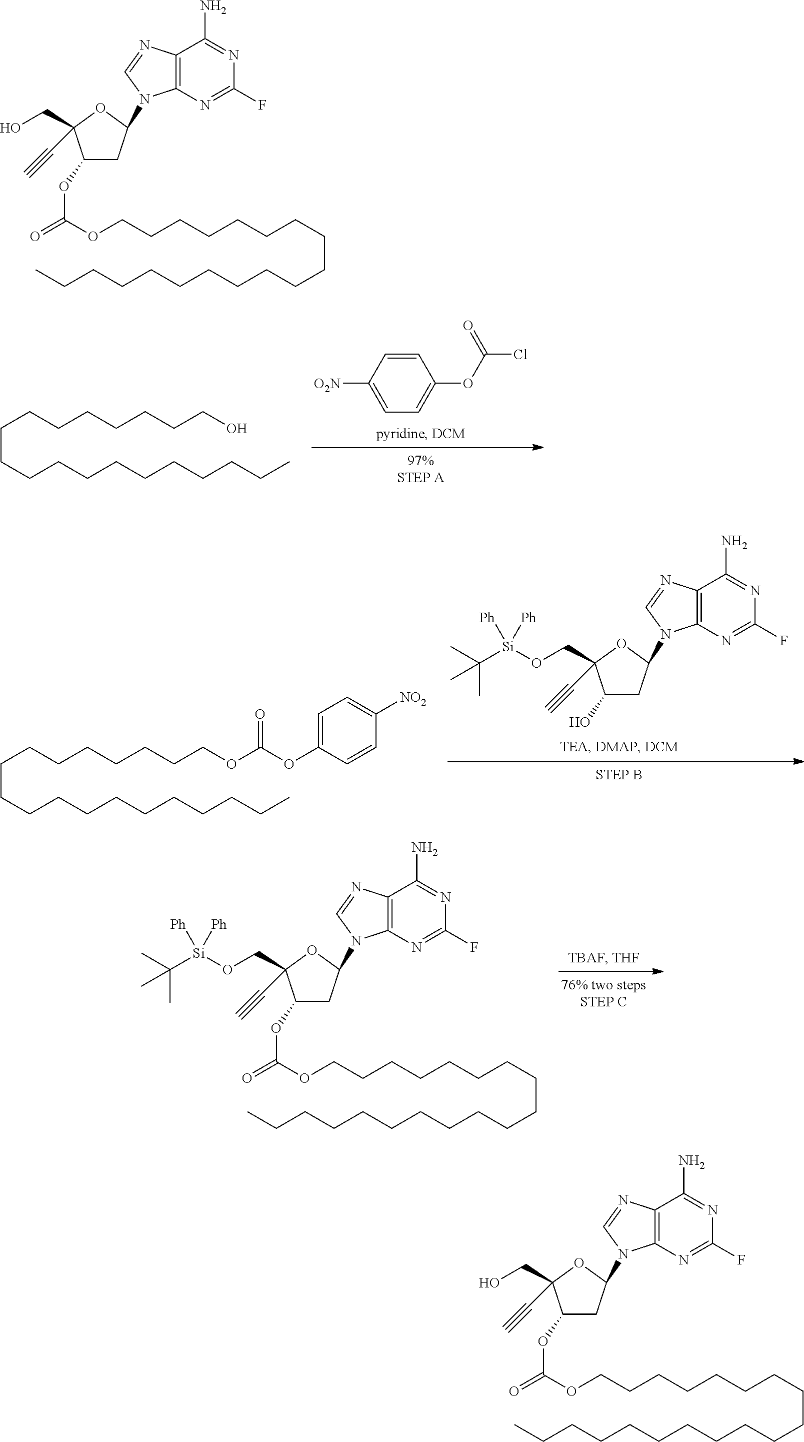

Step A: 4-nitrophenyl nonadecyl carbonate. A mixture of nonadecan-1-ol (530 mg, 1.86 mmol) and 4-nitrophenyl carbonochloridate (451 mg, 2.24 mmol) in DCM (10 mL) was treated with pyridine (3.00 mL, 37.1 mmol) and the mixture was stirred at RT for 1 h. The reaction mixture was concentrated and purified by flash chromatography (silica gel, 100% DCM) to give the desired product (810 mg, 97%) as a white solid. ¹H NMR (400 MHz, CDCl₃) δ 8.30 (d, J=8.1 Hz, 2H), 7.41 (d, J=8.1 Hz, 2H), 4.31 (t, J=6.7 Hz, 2H), 1.74-1.83 (m, 2H), 1.39-1.50 (m, 2H), 1.29 (s, 30H), 0.86-0.96 (m, 3H).

Step B: (2R,3S,5R)-5-(6-amino-2-fluoro-9H-purin-9-yl)-2-(((tert-butyldiphenylsilynoxy)methyl)-2-ethynyltetrahydrofuran-3-yl nonadecyl carbonate. To a stirred solution of (2R,3S,5R)-5-(6-amino-2-fluoro-4,5-dihydro-9H-purin-9-yl)-2-(((tert-butyldiphenylsilyl)oxy)methyl)-2-ethynyltetrahydrofuran-3-ol (150 mg, 0.281 mmol), and DMAP (34.3 mg, 0.281 mmol)) in DCM (5 mL) was added TEA (0.157 mL, 1.12 mmol) followed by 4-nitrophenyl nonadecyl carbonate (152 mg, 0.337 mmol) and the mixture was stirred at RT for 36 h. The reaction mixture was concentrated and purified by preparative TLC (Hex/EtOAc 1:1) to give the desired product as a solid. ¹H NMR (400 MHz, CDCl₃) δ 7.96 (s, 1H), 7.68 (qd, J=3.8, 1.4 Hz, 4H), 7.35-7.48 (m, 6H), 6.49 (t, J=6.7 Hz, 1H), 5.83 (bs, 2H), 5.72 (dd, J=6.9, 4.1 Hz, 1H), 4.22 (td, J=6.8, 2.2 Hz, 2H), 4.09 (d, J=11.0 Hz, 1H), 4.00 (d, J=11.2 Hz, 1H), 2.96 (dt, J=14.0, 7.1 Hz, 1H), 2.80 (ddd, J=13.7, 6.3, 4.1 Hz, 1H), 2.62 (s, 1H), 1.64-1.80 (m, 2H), 1.23-1.43 (m, 32H), 1.07-1.13 (m, 9H), 0.85-0.95 (m, 3H).

Step C: (2R,3S,5R)-5-(6-amino-2-fluoro-9H-purin-9-yl)-2-ethynyl-2-(hydroxymethyl)tetrahydrofuran-3-yl nonadecyl carbonate. To a solution of (2R,3S,5R)-5-(6-amino-2-fluoro-9H-purin-9-yl)-2-(((tert-butyldiphenylsilyl)oxy)methyl)-2-ethynyltetrahydrofuran-3-yl nonadecyl carbonate in THF (5.00 mL) was added TBAF (1 M, THF) (0.422 mL, 0.422 mmol) and the mixture was stirred at RT for 30 min. The mixture was concentrated and the residue purified by flash chromatography (silica gel, DCM/MeOH, 0-5%) to give the desired product as a white solid (129 mg, 76% for two steps). LCMS (ESI) m/z calcd for C₃₂H₅₀FN₅O₅: 603. Found: 604 (M+1)⁺. ¹H NMR (400 MHz, CDCl₃) δ 7.95 (s, 1H), 6.38 (dd, J=9.1, 5.5 Hz, 1H), 5.98 (s, 2H), 5.65 (dd, J=6.1, 1.8 Hz, 1H), 4.19-4.29 (m, 2H), 4.04-4.12 (m, 1H), 3.95-4.00 (m, 1H), 3.24 (ddd, J=13.9, 9.1, 6.3 Hz, 1H), 2.67 (s, 1H), 2.60 (ddd, J=13.9, 5.6, 1.8 Hz, 1H), 1.69-1.77 (m, 2H), 1.27-1.49 (m, 32H), 0.87-0.94 (m, 3H).

### Example 10

### (2R,3S,5R)-5-(6-amino-2-fluoro-9H-purin-9-yl)-2-ethynyl-2-(hydroxymethyl)tetrahydrofuran-3-yl icosyl carbonate

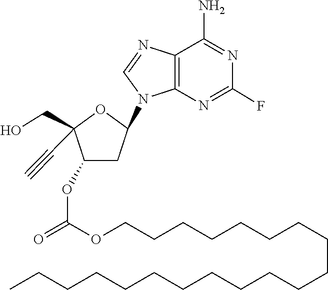 [SMILES: C#C[C@]1(CO)O[C@@H](n2cnc3c(N)nc(F)nc32)C[C@@H]1OC(=O)OCCCCCCCCCCCCCCCCCCCC]

The title compound was prepared as described herein for the synthesis of (2R,3S,5R)-5-(6-amino-2-fluoro-9H-purin-9-yl)-2-ethynyl-2-(hydromethyl)tetrahydrofuran-3-yl nonadecyl carbonate, substituting icosan-1-ol for nonadecan-1-ol is step A. LCMS (ESI) m/z calcd for C₃₃H₅₂FN₅O₅: 617. Found: 618 (M+1)⁺. ¹H NMR (400 MHz, CDCl₃) δ 7.83 (s, 1H), 6.37 (dd, J=9.3, 5.5 Hz, 1H), 6.26 (br s, 2H), 5.66 (dd, J=6.3, 1.3 Hz, 1H), 4.16-4.29 (m, 2H), 4.07 (d, J=12.6 Hz, 1H), 3.97 (d, J=12.6 Hz, 1H), 3.25 (ddd, J=14.0, 9.3, 6.3 Hz, 1H), 2.66 (s, 1H), 2.57 (ddd, J=13.9, 5.5, 1.6 Hz, 1H), 1.67-1.79 (m, 2H), 1.28 (s, 32H) 1.41 (m, Hz, 2H), 0.85-0.96 (m, 3H).

### Example 11

### (((2R,3S,5R)-5-(6-amino-2-fluoro-9H-purin-9-yl)-2-ethynyl-3-hydroxytetrahydrofuran-2-yl)methoxy)methyl hexadecyl carbonate

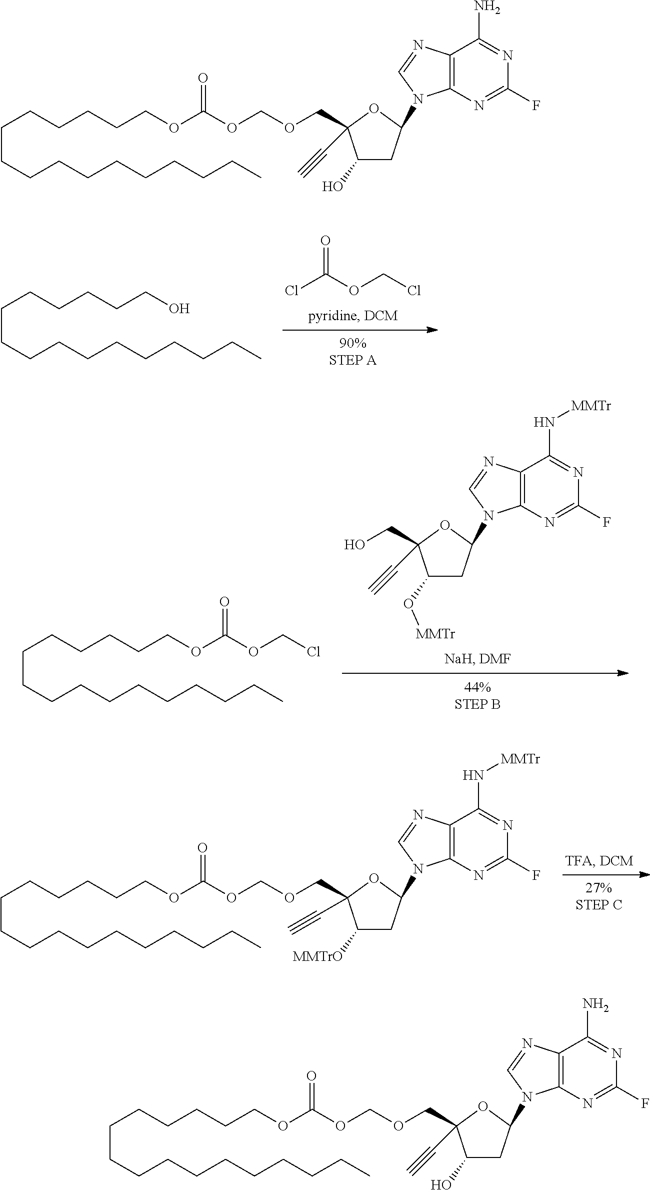

Step A: chloromethyl hexadecyl carbonate. To a stirred solution of chloromethyl carbonochloridate (1.034 mL, 11.63 mmol) in DCM (45 mL) at 0° C. was added dropwise over 10 minutes a solution of hexadecan-1-ol (2.82 g, 11.6 mmol) and pyridine (0.988 mL, 12.2 mmol) in DCM (12 mL). The mixture was stirred at 0° C. for 30 minutes. The mixture was diluted with DCM, washed with 0.5M aqueous HCl, water, saturated aqueous NaHCO₃, and finally brine, dried over Na₂SO₄, filtered and concentrated. The residue was purified on silica gel (0-30% EtOAc/hexanes) to afford the title compound (3.51 g, 90%) as a colorless oil that solidified to a white solid upon standing. ¹H NMR (400 MHz, CDCl₃) δ 5.74 (s, 2H), 4.23 (t, J=6.7 Hz, 2H), 1.77-1.65 (m, 2H), 1.43-1.19 (m, 26H), 0.93-0.86 (m, 3H).

Step B: (((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)methyl hexadecyl carbonate. To a stirred solution of ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methanol (2.0 g, 2.387 mmol) in DMF (22 mL) at 0° C. was added sodium hydride (0.239 g, 5.97 mmol). The mixture was allowed to stir at ambient temperature for 10 minutes. A solution of chloromethyl hexadecyl carbonate (1.199 g, 3.58 mmol) in DMF (4 mL) was added and the mixture was stirred for 15 minutes. Water was added and stirred for several minutes and the mixture was extracted with EtOAc. The combined extracts were washed with water, then brine, dried over Na₂SO₄, filtered and concentrated. The residue was purified on silica gel (0-10% EtOAc/DCM) to afford the title compound (1.20 g, 44%) as a white foam. ¹H NMR (400 MHz, CDCl₃) δ 5.74 (s, 2H), 4.23 (t, J=6.7 Hz, 2H), 1.77-1.65 (m, 2H), 1.43-1.19 (m, 26H), 0.93-0.86 (m, 3H).

Step C: (((2R,3S,5R)-5-(6-amino-2-fluoro-9H-purin-9-yl)-2-ethynyl-3-hydroxytetrahydrofuran-2-yl)methoxy)methyl hexadecyl carbonate. To a solution of (((2R,3S,5R)-2-ethynyl-5-(2-fluoro-6-(((4-methoxphenyl)diphenylmethyl)amino)-9H-purin-9-yl)-3-((4-methoxyphenyl)diphenylmethoxy)tetrahydrofuran-2-yl)methoxy)methyl hexadecyl carbonate (1.40 g, 1.24 mmol) in DCM (50 mL) at 0° C. was slowly added 5% TFA/DCM (500 uL). The mixture was warmed to RT and then stirred ˜4 days at ambient temperature. The mixture was recooled and additional 5% TFA/DCM (500 uL) was added and the mixture was stirred for an additional 14 days. The solution was concentrated to dryness and the residue subjected to flash chromatography twice (silica gel, 0-10% MeOH/DCM) to afford the title compound (197 mg, 27%) as a white solid. LCMS (ESI) m/z calcd for C₃₀H₄₆FN₅O₆: 591.3. Found: 592.4 (M+1)⁺. ¹H NMR (400 MHz, DMSO-d₆) δ 8.24 (s, 1H), 7.80 (br s, 2H), 6.25 (dd, J=4.8, 7.6 Hz, 1H), 5.69 (d, J=5.5 Hz, 1H), 5.26-5.16 (m, 2H), 4.62-4.52 (m, 1H), 4.07-4.02 (m, 2H), 3.90 (d, J=11.2 Hz, 1H), 3.82 (d, J=11.2 Hz, 1H), 3.57 (s, 1H), 2.78-2.69 (m, 1H), 2.54-2.40 (m, 1H, overlapping DMSO peak), 1.60-1.50 (m, 2H), 1.35-1.14 (m, 26H), 0.91-0.80 (m, 3H).

### Example 12

### dodecyl (((2R,3S,5R)-2-ethynyl-5-(2-fluoro-6-heptanamido-9H-purin-9-yl)-3-hydroxytetrahydrofuran-2-yl)methyl) carbonate

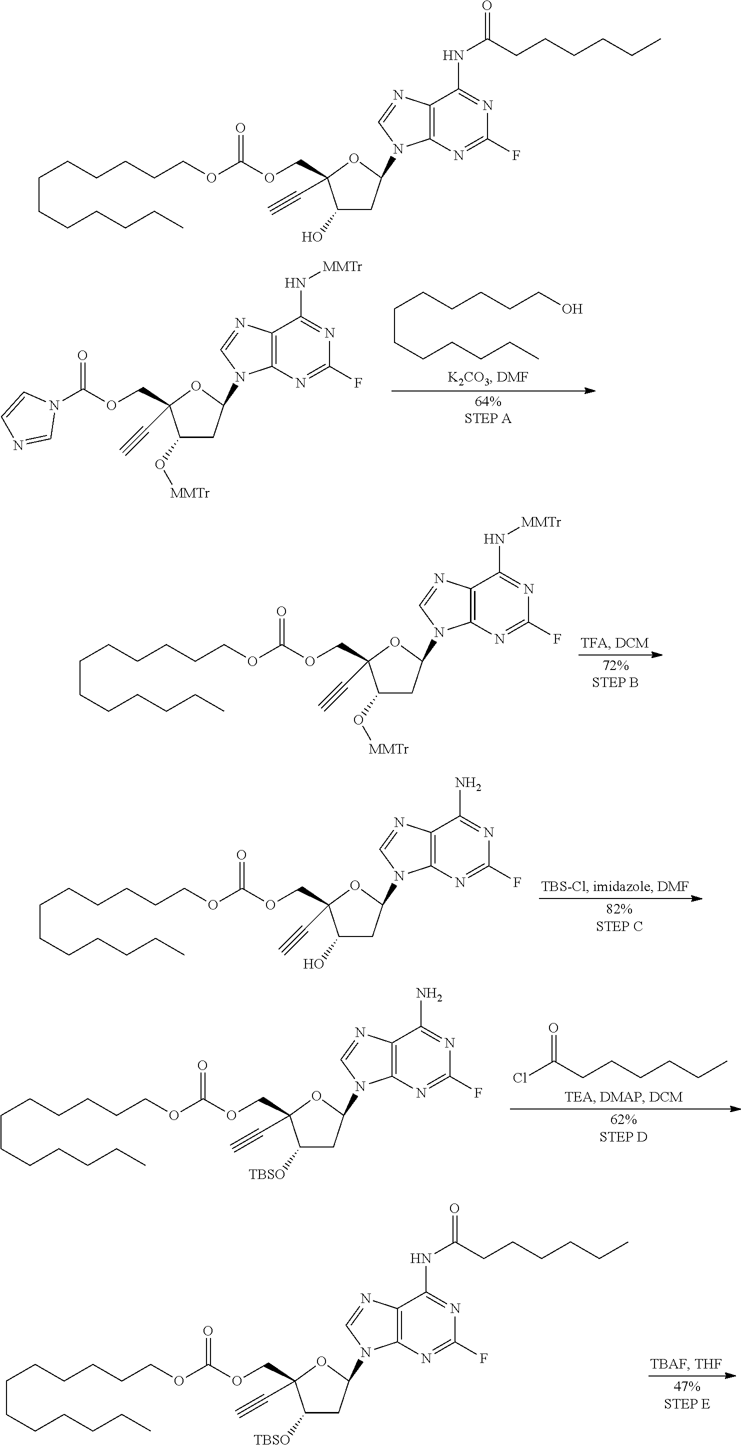

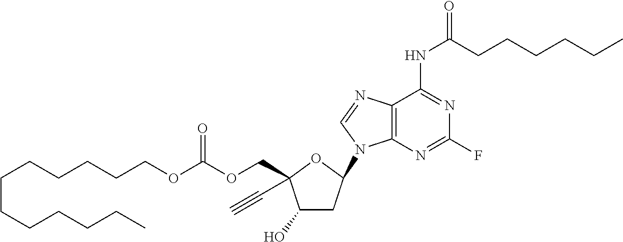 [SMILES: C#C[C@]1(COC(=O)OCCCCCCCCCCCC)O[C@@H](n2cnc3c(NC(=O)CCCCCC)nc(F)nc32)C[C@@H]1O]

Step A: dodecyl ((((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl) carbonate. To a mixture of ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl 1H-imidazole-1-carboxylate (1.00 g, 1.07 mmol) in DMF (10 mL) was added dodecan-1-ol (0.400 g, 2.146 mmol) and potassium carbonate (0.297 g, 2.146 mmol), and the resulting mixture was stirred overnight at RT. LCMS indicated complete reaction. The reaction mixture was diluted with water (20 mL), extracted with EtOAc (3×10 mL), the organic phases were combined, washed with brine (20 mL), dried over Na₂SO₄, and concentrated under vacuum. The mixture was subjected to flash chromatography (silica gel, 1:1 EtOAc/petroleum ether) to give the desired product (730 mg, 63%) as a white solid. LCMS (ESI) m/z calcd for C₆₅H₆₈FN₅O₇: 1050. Found: 1051 (M+1)⁺. ¹H NMR (400 MHz, CDCl₃) δ 7.68 (s, 1H), 7.53 (d, J=8 Hz, 4H), 7.39 (d, J=8 Hz, 2H), 7.32-7.28 (m, 8H), 7.24-7.16 (m, 10H), 6.81-6.77 (m, 4H), 6.17-6.14 (m, 1H), 4.56 (t, J=16 Hz, 1H), 4.30 (d, J=16 Hz, 1H), 4.14-4.09 (m, 1H), 4.04-4.01 (m, 2H), 3.96 (d, J=12 Hz, 1H), 3.76 (d, J=12 Hz, 6H), 2.83 (s, 1H), 2.21-2.14 (m, 1H), 1.80-1.74 (m, 1H), 1.63-1.57 (m, 4H), 1.32-1.22 (m, 16H), 0.89-0.86 (m, 3H).

Step B: ((2R,3S,5R)-5-(6-amino-2-fluoro-9H-purin-9-yl)-2-ethynyl-3-hydroxytetrahydrofuran-2-yl)methyl dodecyl carbonate. To a mixture of dodecyl (((2R,3S,5R)-2-ethynyl-5-(2-fluoro-6-(((4-methoxphenyl)diphenylmethyl)amino)-9H-purin-9-yl)-3-((4-methoxyphenyl)diphenylmethoxy)tetrahydrofuran-2-yl)methyl) carbonate (1.10 g, 1.05 mmol) in DCM (11 mL) was added, TFA (1.2 mL, 16 mmol), and the resulting mixture was stirred for 1 h at RT. LCMS indicated complete reaction. The reaction mixture was added with MeOH (10 mL), then DCM was removed under vacuum. The mixture was subjected to preparative TLC (silica gel, 20:1 DCM/MeOH) to give the desired product (400 mg, 72%) as a white solid. LCMS (ESI) m/z calcd for C₂₅H₃₆FN₅O₅: 505. Found: 506 (M+1)⁺. ¹H NMR (300 MHz, CDCl₃) δ 7.96 (s, 1H), 6.39 (s, 1H), 6.05 (br, 2H), 4.79 (s, 1H), 4.55-4.43 (m, 2H), 4.18-4.12 (m, 2H), 2.99-2.86 (m, 1H), 2.80 (s, 1H), 2.75-2.59 (m, 1H), 1.72-1.58 (m, 2H), 1.35-1.20 (m, 18H), 0.89-0.85 (m, 3H).

Step C: ((2R,3S,5R)-5-(6-amino-2-fluoro-9H-purin-9-yl)-3-((tert-butyldimethylsilyl)oxy)-2-ethynyltetrahydrofuran-2-yl)methyl dodecyl carbonate. To a solution of ((2R,3S,5R)-5-(6-amino-2-fluoro-9H-purin-9-yl)-2-ethynyl-3-hydroxytetrahydrofuran-2-yl)methyl dodecyl carbonate (400 mg, 0.791 mmol) and imidazole (162 mg, 2.37 mmol) in DMF (10 mL) stirred under nitrogen at 0° C. was added a solution of TBS-CI (358 mg, 2.37 mmol) in DMF (1 mL) dropwise during 1 min. The reaction mixture was stirred at RT for 16 hr. LCMS indicated complete reaction. The reaction was diluted with water (200 mL) and the mixture was extracted with EtOAc (3×200 mL). The combined organic layer was concentrated in vacuo. The residue was subjected to flash chromatography (silica gel, 1:1 EtOAc/petroleum ether) to give the desired product (400 mg, 82%) as a white solid. LCMS (ESI) m/z calcd for C₃₁H₅₀FN₅O₅Si: 619. Found: 620 (M+1)⁺. ¹H NMR (300 MHz, CDCl₃) δ 7.93 (s, 1H), 6.35-6.31 (m, 1H), 6.05 (br, 2H), 4.84 (t, J=15 Hz, 1H), 4.50 (d, J=12 Hz, 1H), 4.29 (d, J=12 Hz, 1H), 4.15-4.06 (m, 2H), 2.82-2.74 (m, 1H), 2.67-2.58 (m, 2H), 1.68-1.59 (m, 2H), 1.34-1.19 (m, 18H), 0.96 (s, 9H), 0.87 (t, J=12 Hz, 3H), 0.13 (d, J=6 Hz, 6H).

Step D: ((2R,3S,5R)-3-((tert-butyldimethylsilyl)oxy)-2-ethynyl-5-(2-fluoro-6-heptanamido-9H-purin-9-yl)tetrahydrofuran-2-yl)methyl dodecyl carbonate. To a solution of ((2R,3S,5R)-5-(6-amino-2-fluoro-9H-purin-9-yl)-3-((tert-butyldimethylsilyl)oxy)-2-ethynyltetrahydrofuran-2-yl)methyl dodecyl carbonate (400 mg, 0.645 mmol), DMAP (39.4 mg, 0.323 mmol) and TEA (0.450 mL, 3.23 mmol) in DCM (20 mL) stirred under nitrogen at 0° C. was added a solution of heptanoyl chloride (192 mg, 1.291 mmol) in DCM (2.00 mL) dropwise during 1 min. The reaction mixture was stirred at RT for 3 hr. LCMS indicated complete reaction. The reaction was quenched by pouring into brine (200 mL) and the mixture was extracted with EA (200 mL). The organic layer was concentrated in vacuo. The mixture was subjected to flash chromatography (silica gel, 1:1 EtOAc/petroleum ether) to give the desired product (300 mg, 62%) as a white solid. LCMS (ESI) m/z calcd for C₃₈H₆₂FN₅O₆Si: 731. Found: 732 (M+1)⁺. ¹H NMR (400 MHz, CDCl₃) δ 8.51 (s, 1H), 8.10 (s, 1H), 6.39-6.36 (m, 1H), 4.83 (t, J=16 Hz, 1H), 4.49 (d, J=8 Hz, 1H), 4.30 (d, J=8 Hz, 1H), 4.16-4.05 (m, 2H), 2.98-2.91 (m, 2H), 2.80-2.74 (m, 1H), 2.69-2.62 (m, 2H), 1.79-1.59 (m, 4H), 1.46-1.39 (m, 2H), 1.35-1.20 (m, 28H), 0.93 (s, 9H), 0.14 (d, J=8 Hz, 6H).

Step E: dodecyl (((2R,3S5R)-2-ethynyl)-5-(2-fluoro-6-heptanamido-9H-purin-9-yl)-3-hydroxytetrahydrofuran-2-yl)methyl) carbonate. To a solution of ((2R,3S,5R)-3-((tert-butyldimethylsilyl)oxy)-2-ethynyl-5-(2-fluoro-6-heptanamido-9H-purin-9-yl)tetrahydrofuran-2-yl)methyl dodecyl carbonate (300 mg, 0.410 mmol) in THF (10 mL) stirred under nitrogen at 20° C. was added a solution of TBAF (0.82 mL, 0.82 mmol, 1N in THF) in one charge. The reaction mixture was stirred at RT for 1 h. LCMS indicated complete reaction. The reaction was quenched by pouring into water (200 mL) and the mixture was extracted with EA (200 mL). The organic layer was concentrated in vacuo. The mixture was subjected to preparative RP-HPLC (C18, MeCN/water, 0.05% TFA) to give the desired product (120 mg, 47%) as a white solid. LCMS (ESI) m/z calcd for C₃₂H₄₈FN₅O₆: 617. Found: 618 (M+1)⁺. 1H NMR (400 MHz, CDCl₃) δ 8.54 (s, 1H), 8.11 (s, 1H), 6.43 (t, J=12 Hz, 1H), 4.78 (t, J=12 Hz, 1H), 4.52-4.43 (m, 2H), 4.14 (t, J=12 Hz, 2H), 2.96-2.89 (m, 3H), 2.82 (s, 1H), 2.72-2.65 (m, 1H), 1.79-1.62 (m, 4H), 1.44-1.39 (m, 2H), 1.36-1.25 (m, 22H), 0.92-0.85 (m, 6H).

### Example 13

### ((2R,3S,5R)-5-(6-amino-2-fluoro-9H-purin-9-yl)-2-ethynyl-3-hydroxytetrahydrofuran-2-yl)methyl octadecyl carbonate

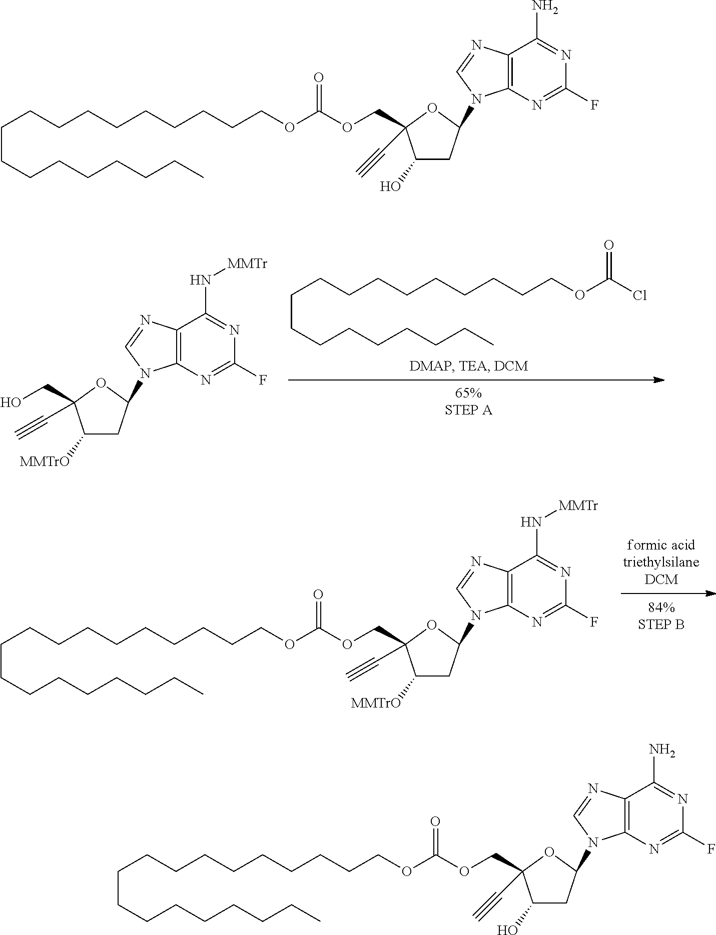

Step A: ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl octadecyl carbonate. To a stirred solution of ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methanol (500 mg, 0.597 mmol) and DMAP (72.9 mg, 0.597 mmol) in DCM (5 mL) at 0° C. was added TEA (0.250 mL, 1.790 mmol), followed by a solution of octadecyl carbonochloridate (397 mg, 1.193 mmol) in DCM (1 mL). The mixture was stirred at 0° C. for 5 minutes and then warmed to ambient temperature and stirred for four hours. Saturated aqueous NaHCO₃ was added and the mixture was extracted with EtOAc. The extracts were washed with brine, dried over Na₂SO₄, filtered and concentrated. The residue was purified on silica gel (0-40% EtOAc/hexanes) to afford the title compound (441 mg, 65%) a colorless residue.

Step B: ((2R,3S,5R)-5-(6-amino-2-fluoro-9H-purin-9-yl)-2-ethynyl-3-hydroxytetrahydrofuran-2-yl)methyl octadecyl carbonate. To a solution of ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methyl octadecyl carbonate (441 mg, 0.389 mmol) in DCM (8 mL) was added dropwise formic acid (2.0 mL, 52.1 mmol) followed by triethylsilane (0.191 mL, 1.19 mmol) and the mixture was allowed to stir at ambient temperature for 2 hours. The mixture was concentrated and then purified on silica gel (0-10% MeOH/DCM) to afford the title compound (193 mg, 84%) as a white solid. LCMS (ESI) m/z calcd for C₃₁H₄₈FN₅O₅: 589.4. Found: 590.4 (M+1)⁺. ¹H NMR (400 MHz, DMSO-d₆) δ 8.17 (s, 1H), 7.80 (br s, 2H), 6.25 (dd, J=4.4, 7.7 Hz, 1H), 5.79 (d, J=5.5 Hz, 1H), 4.71-4.59 (m, 1H), 4.43 (d, J=11.4 Hz, 1H), 4.21 (d, J=11.4 Hz, 1H), 4.03-3.93 (m, 2H), 3.62 (s, 1H), 2.82-2.74 (m, 1H), 2.53-2.43 (m, 1H, overlapping DMSO peak), 1.57-1.47 (m, 2H), 1.31-1.18 (m, 30H), 0.90-0.80 (m, 3H).

### Example 14

### N-(9-((2R,4S,5R)-5-ethynyl-4-hydroxy-5-(hydroxymethyl)tetrahydrofuran-2-yl)-2-fluoro-9H-purin-6-yl)decanamide

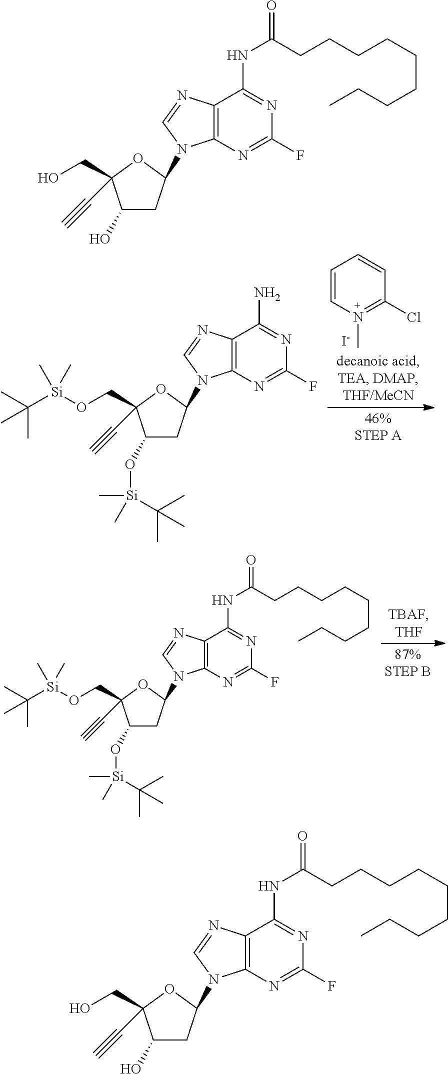

Step A: N-(9-((2R,4S,5R)-4-((tert-butyldimethylsilyl)oxy)-5-(((tert-butyldimethylsilyl)oxy)methyl)-5-ethynyltetrahydrofuran-2-yl)-2-fluoro-9H-purin-6-yl)decanamide. To a solution of 9-((2R,4S,5R)-4-((tert-butyldimethylsilyl)oxy)-5-(((tert-butyldimethylsilyl)oxy) methyl)-5-ethynyltetrahydrofuran-2-yl)-2-fluoro-9H-purin-6-amine (80.0 mg, 0.153 mmol) in MeCN (0.75 mL)/THF (0.75 mL) was added decanoic acid (66.0 mg, 0.383 mmol), 2-chloro-1-methylpyridinium iodide (98.0 mg, 0.383 mmol), TEA (0.128 mL, 0.920 mmol) and DMAP (1.8 mg, 0.015 mmol) and the mixture was stirred at 60° C. for 18 h. The mixture was diluted with DCM and washed with saturated NaHCO₃/water. The organic phase was dried (Na₂SO₄), concentrated and purified on silica gel (EtOAc/hexanes 0-100%) to provide N-(9-((2R,4S,5R)-4-((tert-butyldimethylsilyl)oxy)-5-(((tert-butyldimethylsilyl)oxy)methyl)-5-ethynyltetrahydrofuran-2-yl)-2-fluoro-9H-purin-6-yl)decanamide (48 mg, 46%) as a pale yellow oil. LCMS (ESI) m/z calcd for C34H58FN₅O₄Si2: 675.4. Found: 676.5 (M+1)⁺.

Step B: N-(9-((2R,4S,5R)-5-ethynyl-4-hydroxy-5-(hydroxymethyl)tetrahydrofuran-2-yl)-2-fluoro-9H-purin-6-yl)decanamide. To a solution of N-(9-((2R,4S,5R)-4-((tert-butyldimethylsilyl)oxy)-5-(((tert-butyldimethylsilyl)oxy) methyl)-5-ethynyltetrahydrofuran-2-yl)-2-fluoro-9H-purin-6-yl)decanamide (47 mg, 0.070 mmol) in THF (1.25 mL) at 0° C. was added TBAF (0.174 mL, 0.174 mmol) (1 M/THF). After 18 h at 0° C., acetic acid (9.95 μl, 0.174 mmol) was added and the mixture was adsorbed on silica gel and purified on silica gel (MeOH/dichloromethane 3%) to provide N-(9-((2R,4S,5R)-5-ethynyl-4-hydroxy-5-(hydroxymethyl)tetrahydrofuran-2-yl)-2-fluoro-9H-purin-6-yl)decanamide (27 mg, 87%) as an off-white foam. LCMS (ESI) m/z calcd for C₂₂H₃₀FN₅O₄: 447.2. Found: 448.3 (M+1)⁺. ¹H NMR (400 MHz, METHANOL-d₄) δ 8.53 (s, 1H), 6.46 (dd, J=7.4, 4.3 Hz, 1H), 3.84-3.92 (m, 1H), 3.75-3.82 (m, 1H), 3.11 (s, 1H), 2.84 (ddd, J=13.4, 7.0, 4.4 Hz, 1H), 2.62-2.75 (m, 3H), 1.76 (t, J=7.4 Hz, 2H), 1.23-1.52 (m, 13H), 0.86-0.98 (m, 3H).

### Example 15

### N-(9-((2R,4S,5R)-5-ethynyl-4-hydroxy-5-(hydroxymethyl)tetrahydrofuran-2-yl)-2-fluoro-9H-purin-6-yl)tetradecanamide

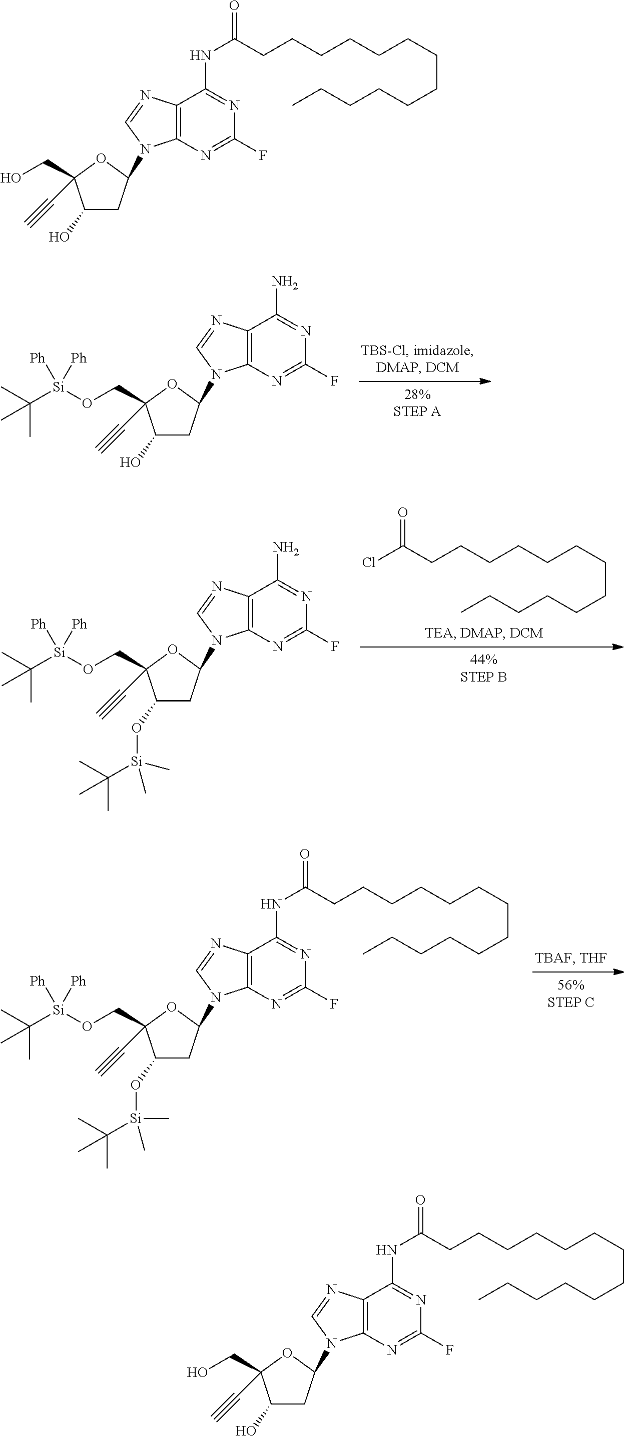 [SMILES: C#C[C@]1(CO)O[C@@H](n2cnc3c(NC(=O)CCCCCCCCCCCCC)nc(F)nc32)C[C@@H]1O ; C#C[C@]1(CO[Si](c2ccccc2)(c2ccccc2)C(C)(C)C)O[C@@H](n2cnc3c(N)nc(F)nc32)C[C@@H]1O ; C#C[C@]1(CO[Si](c2ccccc2)(c2ccccc2)C(C)(C)C)O[C@@H](n2cnc3c(N)nc(F)nc32)C[C@@H]1O[Si](C)(C)C(C)(C)C ; CCCCCCCCCCCCCC(=O)Cl ; C#C[C@]1(CO[Si](c2ccccc2)(c2ccccc2)C(C)(C)C)O[C@@H](n2cnc3c(NC(=O)CCCCCCCCCCCCC)nc(F)nc32)C[C@@H]1O[Si](C)(C)C(C)(C)C ; C#C[C@]1(CO)O[C@@H](n2cnc3c(NC(=O)CCCCCCCCCCCCC)nc(F)nc32)C[C@@H]1O]

Step A: 9-((2R,4S,5R)-4-((tert-butyldimethylsilyl)oxy)-5-(((tert-butyldiphenylsilyl)oxy)methyl)-5-ethynyltetrahydrofuran-2-yl)-2-fluoro-9H-purin-6-amine. A solution of (2R,3S,5R)-5-(6-amino-2-fluoro-9H-purin-9-yl)-2-(((tert-butyldiphenylsilyl)oxy)methyl)-2-ethynyltetrahydrofuran-3-ol (195 mg, 0.367 mmol) and imidazole (49.9 mg, 0.734 mmol) in DMF (3.2 mL) was added TBS-Cl (71.9 mg, 0.477 mmol) at ambient temperature and then mixture was allowed to stir for ˜1 hour. LCMS indicated no change. DMAP (5 mg, 0.041 mmol) was added and the mixture was allowed to stir for 5 days. LCMS indicated no desired product had formed. The mixture was diluted with EtOAc, washed with water, then brine, dried over Na₂SO₄, filtered and concentrated to a white solid. The residue was suspended in DCM (4.0 mL), and treated sequentially with imidazole (49.9 mg, 0.734 mmol), DMAP (44.8 mg, 0.367 mmol) and finally TBS-Cl (71.9 mg, 0.477 mmol). The mixture was allowed to stir at ambient temperature overnight. LCMS indicated ˜35% conversion to the desired product. The reaction mixture was diluted with EtOAc, washed with 0.1N HCl, followed by saturated aqueous NaHCO₃, and finally brine, dried over Na₂SO₄, filtered and concentrated. The residue was purified on silica gel (0-30% EtOAc/DCM) to afford the title compound (66 mg, 28%) as a white solid. LCMS (ESI) m/z calcd for C₃₄H₄₄FN₅O₃Si₂: 645.3. Found: 646.5 (M+1)⁺.

Step B: N-(9-((2R,4S,5R)-4-((tert-butyldimethylsilyl)oxy)-5-(((tert-butyldiphenylsilyl)oxy)methyl)-5-ethynyltetrahydrofuran-2-yl)-2-fluoro-9H-purin-6-yl)tetradecanamide. To a solution of 9-((2R,4S,5R)-4-((tert-butyldimethylsilyl)oxy)-5-(((tert-butyldiphenylsilyl)oxy)methyl)-5-ethynyltetrahydrofuran-2-yl)-2-fluoro-9H-purin-6-amine (66 mg, 0.102 mmol), TEA (0.028 mL, 0.204 mmol) and DMAP (12.48 mg, 0.102 mmol) in DCM (1.5 mL) at 0° C. was added a solution of tetradecanoyl chloride (0.033 mL, 0.123 mmol) in DCM (320 uL) and the mixture was allowed to stir at ambient temperature overnight. The mixture was concentrated and then purified on silica gel (0-30% EtOAc/hexanes) to afford the title (38 mg, 44%) compound as a colorless residue.

Step C: N-(9-((2R,4S,5R)-5-ethynyl-4-hydroxy-5-(hydroxymethyl)tetrahydrofuran-2-yl)-2-fluoro-9H-purin-6-yl)tetradecanamide. To a solution of N-(9-((2R,4S,5R)-4-((tert-butyldimethylsilyl)oxy)-5-(((tert-butyldiphenylsilyl)oxy)methyl)-5-ethynyltetrahydrofuran-2-yl)-2-fluoro-9H-purin-6-yl)tetradecanamide (38 mg, 0.044 mmol) in THF (0.6 mL) was added TBAF, 1M solution in THF (0.111 mL, 0.111 mmol) and the mixture was allowed to stir at ambient temperature for 30 minutes. The mixture was concentrated and then purified on silica gel (0-50% EtOAc/DCM) to afford the title compound (12.5 mg, 56%) as a white solid. LCMS (ESI) m/z calcd for C₂₆H₃₈FN₅O₄: 503.3. Found: 504.4 (M+1)⁺. ¹H NMR (400 MHz, DMSO-d₆) δ 10.97 (s, 1H), 8.62 (s, 1H), 6.35 (dd, J=4.5, 7.4 Hz, 1H), 5.59 (d, J=5.5 Hz, 1H), 5.30-5.19 (m, 1H), 4.69-4.51 (m, 1H), 3.70-3.63 (m, 1H), 3.61-3.55 (m, 1H), 3.53 (s, 1H), 2.81-2.73 (m, 1H), 2.61-2.46 (m, 3H, overlapping DMSO peak), 1.67-1.53 (m, 2H), 1.38-1.16 (m, 20H), 0.94-0.79 (m, 3H).

### Anti-HIV Activity

### PSV Assay

A pseudotyped virus assay (PSV) was used to assess the potency of the HIV inhibitor. Replication defective virus was produced by co-transfection of a plasmid containing an NL4-3 provirus [containing a mutation in the envelope open reading frame (ORF) and a luciferase reporter gene replacing the nef ORF] and a CMV-promoter expression plasmid containing an ORF for various HIV gp160 envelope clones. The harvested virus was stored at −80C in small aliquots and the titer of the virus measured to produce a robust signal for antiviral assays.

The PSV assay was performed by using U373 cells stably transformed to express human CD4, the primary receptor for HIV entry and either human CXCR4 or human CCR5 which are the co-receptors required for HIV entry as target cells for infection. Molecules of interest (including, but not limited to small molecule inhibitors of HIV, neutralizing antibodies of HIV, antibody-drug conjugate inhibitors of HIV, peptide inhibitors of HIV, and various controls) are capable of being diluted into tissue culture media and diluted via serial dilution to create a dose range of concentrations, and this was carried out for Example 1. This dose-range was applied to U373 cells and the pre-made pseudotyped virus added. The amount of luciferase signal produced after 3 days of culture was used to reflect the level of pseudotyped virus infection. An IC₅₀, or the concentration of inhibitor required to reduce PSV infection by 50% from the infection containing no inhibitor was calculated. Assays to measure cytotoxity were performed in parallel to ensure the antiviral activity observed for an inhibitor was distinguishable from reduced target cell viability. IC₅₀ values were determined from a 10 point dose response curve using 3-4-fold serial dilution for each compound, which spans a concentration range >1000 fold.

These values are plotted against the molar compound concentrations using the standard four parameter logistic equation:

y=((Vmax *x\hat{ }n)/(K\hat{ }n+x\hat{ }n))+Y2

where:

Y2=minimum y n=slope factor

Vmax=maximum y x=compound concentration [M]

K=EC₅₀

The resulting data is shown in Tables 3 and 4.

TABLE 3

| | WT IC50 | M184V IC50 |
|---|---|---|
| Example | (μM) | (μM) |
| EFdA | 0.0065 | 0.0351 |
| 1 | 0.0085 | 0.0498 |
| 2 | 0.0095 | 0.0600 |
| 3 | 1.5394 | 6.2926 |
| 4 | 0.0025 | 0.0109 |
| 5 | 0.0195 | 0.1123 |
| 6 | 0.0034 | 0.0111 |
| 7 | 13.3464 | 15.0000 |

TABLE 4

| | WT IC50 | M184V IC50 |
|---|---|---|
| Example | (μM) | (μM) |
| 8 | 0.0289 | 0.1852 |
| 9 | 2.0000 | 19.3600 |
| 10 | 5.4010 | >25 |
| 11 | 0.0065 | 0.0190 |
| 13 | 0.0104 | 0.0411 |
| 14 | 0.0018 | 0.0061 |
| 15 | 0.0014 | 0.0074 |

### Antiviral Persistence Assay

The PSV assay was adapted to determine the antiviral persistence of each compound. This assay evaluates the ability of each compound to remain active in cells for two days i.e. prevent PSV infection of cells in a dose dependent manner, 48 h after the removal of compound. Duplicate plates of U373 cells were treated with a serial dilution of small molecule inhibitors for 6 h at 37° C. Compounds were removed from cells by washing twice cells with 1×PBS. For baseline group (i.e. immediately after washing or 0 h), cells were infected with prepared PSVs and cultured for three days. For experimental group (48 h), the culture medium is added to the washed cells and the plate incubated at 37° C. for 48 h. After two days of culture, the prepared PSVs were added to the cells and the mixture cultured for three days. The amount of luciferase signal produced after culture was used to reflect the level of pseudo typed virus infection in the baseline group (0 h) and experimental group (48 h) for each compound. An IC₅₀, or the concentration of inhibitor required to reduce PSV infection by 50% from the infection containing no inhibitor was calculated. The persistence index, which is the ratio of the IC₅₀ determined at 48 and 0 h is presented in Table 2 as well as the fold change of the persistence index relative to EFdA [(2R,3S,5R)-5-(6-amino-2-fluoro-9H-purin-9-yl)-2-ethynyl-2-(hydroxymethyl)tetrahydrofuran-3-ol].

Statistical analysis and graphing of the data were performed in JMP 13.2.1 (SAS Institute, Cary, N.C.). A four-parameter logistic Hill Model was fit to % Inhibition and log₁₀ concentration values, separately for each compound, time point and run. A pilot experiment included 2 independent experimental runs and a later follow-up experiment included 4 runs. Quality control criteria based on R² and 95% confidence interval ranges of all four parameter estimates were used to exclude curves with poor fits. Using inverse prediction, log₁₀ concentrations were obtained that correspond to 50% Inhibition (log₁₀IC50*) and the log₁₀ persistence index was calculated for each compound and run using the following formula: log₁₀ persistence Index=log₁₀IC50*_{48 hrs}−log₁₀IC50*_{0 hrs}. Next, a linear mixed effects model was fit on log₁₀ persistence index values with a fixed effect for compound and a random effect for experimental run, followed by post hoc contrasts to compare the log₁₀ persistence index of the positive control EFdA to the log₁₀ persistence index of other test compounds. The estimated LSMeans and differences were then back-transformed via 10^{Estimate} to the original scale and reported as persistence index and fold change respectively. Raw p-values were reported. Antiviral persistence data for representative examples and EFdA are shown in Table 5.

TABLE 5

| | WT IC₅₀ | WT IC₅₀ | | Fold | |
|---|---|---|---|---|---|
| | (μM) | (μM) | Persistence | Change | |
| Example | at t = 0 h | at t = 48 h | Index | vs EFdA | p-Value |
| EFdA | 0.0074 | 0.3156 | 42.9 | 1.00 | — |
| 2 | 0.0195 | 0.5321 | 29.9 | 1.44 | 0.5990 |
| 4 | 0.0011 | 0.0219 | 19.1 | 2.25 | 0.2047 |
| 6 | 0.0021 | 0.0986 | 47.7 | 0.90 | 0.8645 |

### Rat Pharmacokinetics Data for Compounds 11 and 13 (Examples 11 and 13)

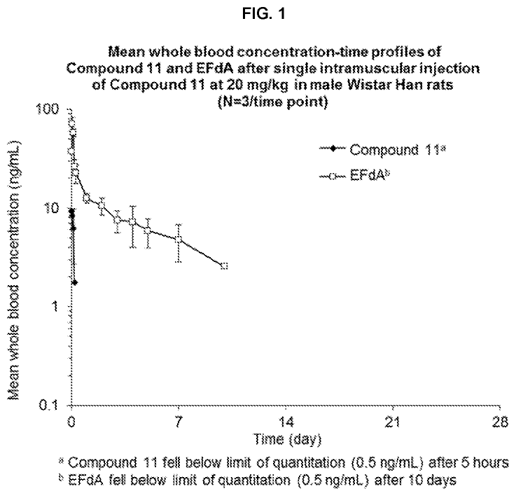

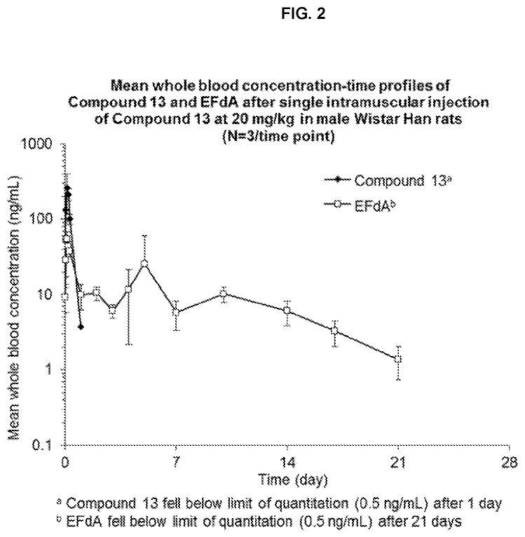

The pharmacokinetics of Compounds 11 and 13 after a single intramuscular (IM) injection were evaluated in Male Wistar Han rats. The test compound was suspended in a 2% P407, 2% PEG3350, 3.5% Mannitol formulation at 10 mg/mL concentration. A single dose of 20 mg/kg of test compound was injected (2 mL/kg) IM into the right gastrocnemius muscle (n=3). Blood samples were collected via a lateral tail vein at the following time points: Day 1 [30 min, 1 h, 3 h, 5 h, 7 h], Days 2-5, Days 7, 10, 14, 17, 21, 24, 28, 31, 35, 38, 42, 45, 49, 52, 56, 59, 63, 66 and 70, etc. To evaluate test compound and EFdA concentrations, approximately 150 μL of blood was collected into a NaFL/Na2EDTA tube. Exactly 150 μL of blood was then pipetted into a new tube, mixed with 150 μL of 100 mM ammonium acetate pH4, (some of the prodrugs required mixing with 1.5 μL of FA as stabilizer), vortexed, immediately frozen on dry ice and stored at −80° C. until analysis. For analysis of test compound and EFdA, frozen blood samples were thawed and mixed with 200 μL of internal standard solution (20 ng/mL Glipizide in acetonitrile), vortexed for 10 min at 750 rpm and centrifuged at 6000 rpm for 10 min. The supernatants were then analyzed by UPLC/MS-MS (Triple Quad™ 6500+). Pharmacokinetic parameters were evaluated using a non-compartmental model of the non-compartmental analysis tool, Pharsight Phoenix WinNonlin® 6.4 software. Results are illustrated in FIGS. 1 and 2.

What is claimed is:

1. A compound of the formula (I): 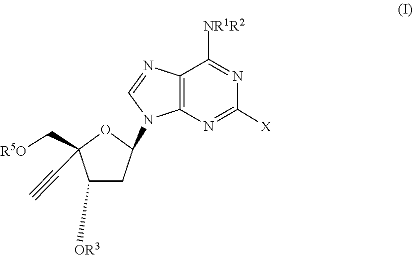
wherein:
R¹ and R² are each independently selected from the group consisting of H and —C(═O)—O—(C₁-C₆)alkyl
R³ is selected from the group consisting of H and —C(═O)—O—R⁴ wherein R⁴ is (C₁-C₆) alkyl;
R⁵ is selected from the group consisting of H, 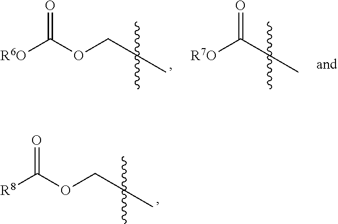
wherein R⁶, R⁷ and R⁸ are independently selected from the group consisting of (C₁-C₂₀)alkyl; and
X is selected from the group consisting of NH₂, F and Cl;
wherein when R³ and R⁵ are each H and X is F or Cl, both R¹ and R² cannot be H;
or a pharmaceutically acceptable salt thereof.

2. The compound according to claim 1, wherein R¹ and R² are each H.

3. The compound according to claim 1, wherein R¹ is H and R² is —(C═O)—O—(C₁-C₆)alkyl.

4. The compound according to claim 1, wherein X is F.

5. The compound according to claim 1, wherein R³ is H.

6. The compound according to any of claim 1, wherein R³ is —C(═O)—O—R⁴.

7. The compound according to claim 1, wherein R⁵ is H.

8. The compound according to claim 1, wherein R⁵ is

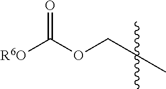

9. The compound according to any of claim 1, wherein R³ is

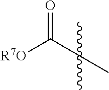

10. A compound selected from the group consisting of:

| Example | Structure | Chemical Name |
|---|---|---|
| 1 | 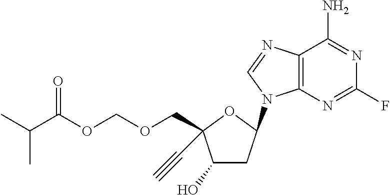 [SMILES: C#C[C@]1(COCOC(=O)C(C)C)O[C@@H](n2cnc3c(N)nc(F)nc32)C[C@@H]1O] | (((2R,35,5R)-5-(6-amino-2- fluoro-9H-purin-9-yl)-2-ethynyl- 3-hydroxytetrahydrofuran-2- yl)methoxy)methyl isobutyrate |
| 2 | 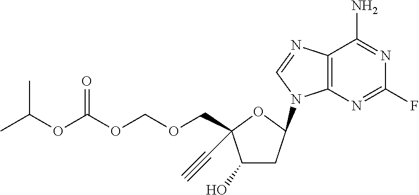 [SMILES: C#C[C@]1(COCOC(=O)OC(C)C)O[C@@H](n2cnc3c(N)nc(F)nc32)C[C@@H]1O] | (((2R,35,5R)-5-(6-amino-2- fluoro-9H-purin-9-yl)-2-ethynyl- 3-hydroxytetrahydrofuran-2- yl)methoxy)methyl isopropyl carbonate |
| 3 | 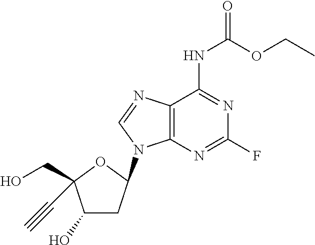 [SMILES: C#C[C@]1(CO)O[C@@H](n2cnc3c(NC(=O)OCC)nc(F)nc32)C[C@@H]1O] | ethyl (9-((2R,45,5R)-5-ethynyl- 4-hydroxy-5- (hydroxymethyl)tetrahydrofuran- 2-yl)-2-fluoro-9H-purin-6- yl)carbamate |
| 4 | 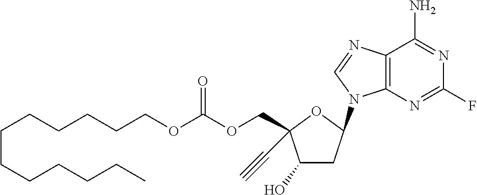 [SMILES: C#C[C@]1(COC(=O)OCCCCCCCCCCCC)O[C@@H](n2cnc3c(N)nc(F)nc32)C[C@@H]1O] | ((2R,35,5R)-5-(6-amino-2- fluoro-9H-purin-9-yl)-2-ethynyl- 3-hydroxytetrahydrofuran-2- yl)methyl dodecyl carbonate |
| 5 | 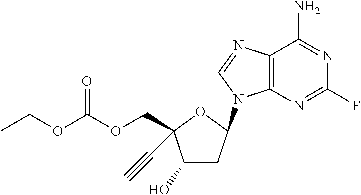 [SMILES: C#C[C@]1(COC(=O)OCC)O[C@@H](n2cnc3c(N)nc(F)nc32)C[C@@H]1O] | ((2R,35,5R)-5-(6-amino-2- fluoro-9H-purin-9-yl)-2-ethynyl- 3-hydroxytetrahydrofuran-2- yl)methyl ethyl carbonate |
| 6 | 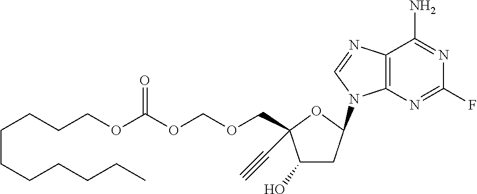 [SMILES: C#C[C@]1(COCOC(=O)OCCCCCCCCCC)O[C@@H](n2cnc3c(N)nc(F)nc32)C[C@@H]1O] | (((2R,3S,5R)-5-(6-amino-2- fluoro-9H-purin-9-yl)-2-ethynyl- 3-hydroxytetrahydrofuran-2- yl)methoxy)methyl decyl carbonate |
| 7 | 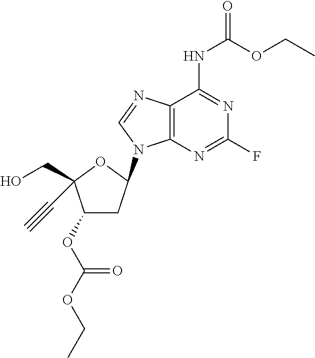 [SMILES: C#C[C@]1(CO)O[C@@H](n2cnc3c(NC(=O)OCC)nc(F)nc32)C[C@@H]1OC(=O)OCC] | ethyl (9-((2R,4S,5R)-4- ((ethoxycarbonyl)oxy)-5- ethynyl-5- (hydroxymethyl)tetrahydrofuran- 2-yl)-2-fluoro-9H-purin-6- yl)carbamate |
| 8 | 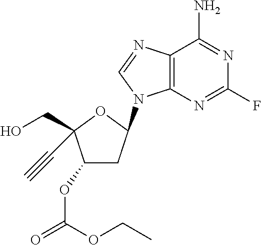 [SMILES: C#C[C@]1(CO)O[C@@H](n2cnc3c(N)nc(F)nc32)C[C@@H]1OC(=O)OCC] | (2R,3S,5R)-5-(6-amino-2-fluoro- 9H-purin-9-yl)-2-ethynyl-2- (hydroxymethyl)tetrahydrofuran- 3-yl ethyl carbonate |
| 9 | 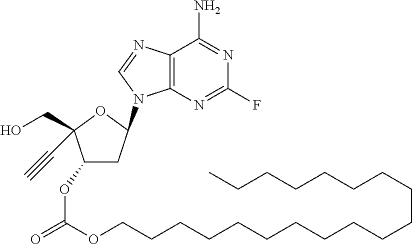 [SMILES: C#C[C@]1(CO)O[C@@H](n2cnc3c(N)nc(F)nc32)C[C@@H]1OC(=O)OCCCCCCCCCCCCCCCCCCC] | (2R,3S,5R)-5-(6-amino-2-fluoro- 9H-purin-9-yl)-2-ethynyl-2- (hydroxymethyl)tetrahydrofuran- 3-yl nonadecyl carbonate |
| 10 | 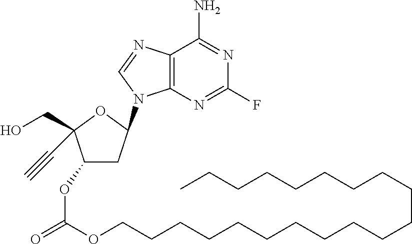 [SMILES: C#C[C@]1(CO)O[C@@H](n2cnc3c(N)nc(F)nc32)C[C@@H]1OC(=O)OCCCCCCCCCCCCCCCCCCCC] | (2R,3S,5R)-5-(6-amino-2-fluoro- 9H-purin-9-yl)-2-ethynyl-2- (hydroxymethyl)tetrahydrofuran- 3-yl icosyl carbonate |
| 11 | 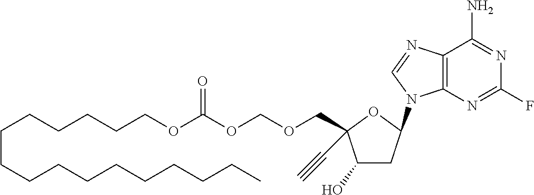 [SMILES: C#C[C@]1(COCOC(=O)OCCCCCCCCCCCCCCCC)O[C@@H](n2cnc3c(N)nc(F)nc32)C[C@@H]1O] | (((2R,3S,5R)-5-(6-amino-2- fluoro-9H-purin-9-yl)-2-ethynyl- 3-hydroxytetrahydrofuran-2- yl)methoxy)methyl hexadecyl carbonate |
| 12 | 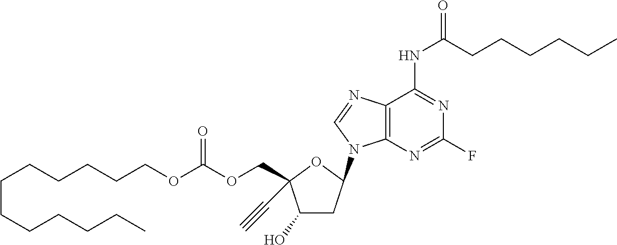 [SMILES: C#C[C@]1(COC(=O)OCCCCCCCCCCCC)O[C@@H](n2cnc3c(NC(=O)CCCCCC)nc(F)nc32)C[C@@H]1O] | dodecyl (((2R,3S,5R)-2-ethynyl- 5-(2-fluoro-6-heptanamido-9H- purin-9-yl)-3- hydroxytetrahydrofuran-2- yl)methyl) carbonate |
| 13 | 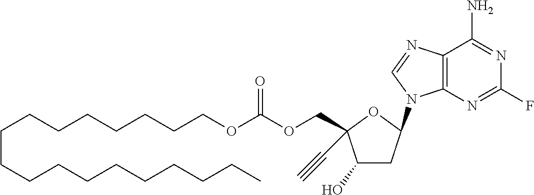 [SMILES: C#C[C@]1(COC(=O)OCCCCCCCCCCCCCCCCCC)O[C@@H](n2cnc3c(N)nc(F)nc32)C[C@@H]1O] | ((2R,3S,5R)-5-(6-amino-2- fluoro-9H-purin-9-yl)-2-ethynyl- 3-hydroxytetrahydrofuran-2- yl)methyl octadecyl carbonate |
| 14 | 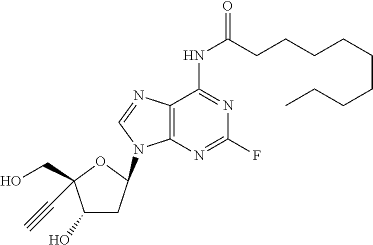 [SMILES: C#C[C@]1(CO)O[C@@H](n2cnc3c(NC(=O)CCCCCCCCC)nc(F)nc32)C[C@@H]1O] | N-(9-((2R,4S,5R)-5-ethynyl-4- hydroxy-5- (hydroxymethyl)tetrahydrofuran- 2-yl)-2-fluoro-9H-purin-6- yl)decanamide |
| 15 | 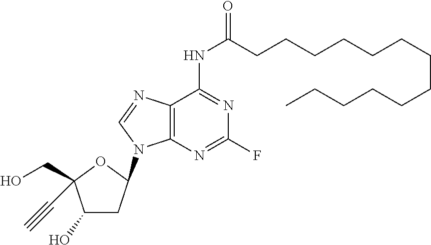 [SMILES: C#C[C@]1(CO)O[C@@H](n2cnc3c(NC(=O)CCCCCCCCCCCCC)nc(F)nc32)C[C@@H]1O] | N-(9-((2R,4S,5R)-5-ethynyl-4- hydroxy-5- (hydroxymethyl)tetrahydrofuran- 2-yl)-2-fluoro-9H-purin-6- yl)tetradecanamide |

and pharmaceutically acceptable salts thereof.

11. A pharmaceutical composition comprising a compound according to claim 1, or a pharmaceutically acceptable salt thereof, and a pharmaceutically acceptable excipient.

12. The composition of claim 11, wherein the composition is present in parenteral form.

13. The composition of claim 11, wherein the composition is in a tablet form.

14. A method of treating an HIV infection in a subject comprising administering to the subject a compound of claim 1, or a pharmaceutically acceptable salt thereof.

15. (canceled)

16. A method of preventing an HIV infection in a subject at risk for developing an HIV infection, comprising administering to the subject a compound of claim 1, or a pharmaceutically acceptable salt thereof.

17-21. (canceled)
